SEQUENCE LISTING (1 sequence)

SEQ ID NO: 1 - protein, 208 aa, Artificial Sequence
  Synthetic
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPEQEEMTKNQVSLRCLVKGFYPSDIAVEWESNGQPENNYKTTKPVLDSDGSFRLESRLTVDKSRWQEGNVFSCSVMHEACSYHLCKSLSLSL

(19) United States
(12) Patent Application Publication
(10) Pub. No.: US 2024/0239898 A1
(43) Pub. Date: Jul. 18, 2024

(54) MODIFIED FCRN BINDING FRAGMENTS WITH IMPROVED HALF-LIFE

(71) Applicant: MedImmune, LLC, Gaithersburg, MD (US)

(72) Inventors: Vaheh Yuni OGANESYAN, Gaithersburg, MD (US); Lu SHAN, Gaithersburg, MD (US)

(21) Appl. No.: 18/559,910

(22) PCT Filed: May 9, 2022
(86) PCT No.: PCT/US2022/072205

§ 371 Date: Nov. 9, 2023

Related U.S. Application Data
(60) Provisional application No. 63/186,445, filed on May 10, 2021.

(51) Int. Cl. C07K 16/28; A61K 47/68

(52) U.S. Cl. CPC C07K 16/283; A61K 47/68; C07K 2317/31; C07K 2317/524; C07K 2317/526; C07K 2317/55; C07K 2317/622; C07K 2317/94

(57) ABSTRACT

The present disclosure relates to modified FcRn binding fragments with improved half-life, specifically fusion proteins and polypeptides comprising said modified FcRn binding fragments, and methods of manufacturing said fusion proteins and their use in methods of treatment.

### TECHNICAL FIELD

The present disclosure relates to modified FcRn binding fragments with improved half-life, specifically fusion proteins and polypeptides comprising said modified FcRn binding fragments, and methods of manufacturing said fusion proteins and their use in methods of treatment.

### BACKGROUND

The immunoglobulin Fc region is a homodimer consisting of two sets of CH2 and CH3 domains and has been exploited to generate two-arm protein fusions with high expression yields, simplified purification processes and extended serum half-life. However, attempts to generate one-arm fusion proteins with monomeric Fc, with one set of CH2 and CH3 domains, are often plagued with challenges such as weakened binding to FcRn or partial monomer formation.

Monovalent versions of Fc fusion proteins (Alprolix-coagulation factor IX fusion, Eloctate—factor VIII fusion) or monovalent antibodies (Onartuzumab—anti-cMet one-arm mAb) that have advanced to the clinic utilize an Fc region that is engineered to form a heterodimer, either with tethering or “knobs-into-holes” technology. These, along with other heterodimeric Fc technologies, rely on robust purification processes to remove undesired chain pairing and achieve a homogeneous fusion protein.

To search for an alternative approach aimed at simplifying product development, there has been extensive effort in engineering fusion protein platforms with a monomeric Fc modality consisting of only one set of CH2 and CH3 domains, either through weakening the interactions or by generating steric hindrances with the addition of glycans at the CH3-CH3 dimer interface in the Fc. So far most of these approaches have encountered challenges in several aspects, including solubility and stability, loss of FcRn binding, or lack of homogeneity. Additionally, many of the previously engineered monomeric Fc molecules were observed by dynamic light scattering to have a tendency for aggregation, highlighting the challenge of stabilizing the monomeric conformation after weakening the homodimer interface. Among the engineered monomeric Fc modalities, only two molecules have been reported to have crystal structures with demonstrable homogeneity and stability. One of these is a monomer that is stabilized by the addition of a glycosylation site that blocks the CH3-CH3 interaction. The other is a monomeric Fc derived from an IgG4 phage library that was rationally designed on the basis of previous findings (Shan et al (2016) PLoS ONE 11(8): e0160345). However, it has been noted that the second platform can oligomerise at high concentrations, which may undermine its utility for pharmaceutical products that require delivery at high concentrations.

Due to the increasing precedence of approved biologic therapeutics and the expansion of their modes of administrations and mechanisms of use, there remains a desire to develop a robust platform of monovalent versions of Fc fusion proteins. For example, gene therapy applications and inhalable products are both rich development areas for biologic therapeutic modalities. However, both have size constraints with respect to the ability to package DNA encoding the therapeutic, or optimum size for effective biodistribution in the lung. In addition, generating smaller molecules with similar pharmacokinetic or pharmacodynamic properties to monoclonal antibodies is generally attractive to improve commercial manufacturing yields and reduce the cost of goods.

Accordingly, there remains a need to provide monovalent versions of Fc fusion proteins.

### SUMMARY OF THE DISCLOSURE

The disclosure relates to the surprising discovery that modifications in the FcRn-CH3 dimerisation interface improves monomerization of FcRn binding fragments. As such, these FcRn binding fragments have improved developability characteristics when used in fusion proteins, polypeptides or FcRn binding fragment-non-protein agent conjugates (referred to herein as “molecules”) requiring or desiring of monovalent FcRn binding fragments derived from Fc regions.

Accordingly, the disclosure provides a fusion protein comprising a FcRn binding fragment of an Fc region of an IgG molecule, wherein the FcRn binding fragment comprises: phenylalanine (F) at position 351; arginine (R), lysine (K), aspartate (D), glutamate (E), phenylalanine (F), tyrosine (Y), proline (P), glycine (G), leucine (L) or methionine (M) at position 354; arginine (R) at position 366; lysine (K) at position 395; arginine (R) at position 405; and glutamate (E) at position 407, wherein the amino acid numbering is according to the EU index. It has been found that substituting wild-type residues at each of these positions with these amino acids improve monomer stability of FcRn binding fragments.

In another aspect, the disclosure provides a polypeptide comprising at least a FcRn binding fragment of an Fc region of an IgG molecule, wherein the FcRn binding fragment comprises: phenylalanine (F) at position 351; arginine (R), lysine (K), aspartate (D), glutamate (E), phenylalanine (F), tyrosine (Y), proline (P), glycine (G), leucine (L) or methionine (M) at position 354; arginine (R) at position 366; lysine (K) at position 395; arginine (R) at position 405; and glutamate (E) at position 407, wherein the amino acid numbering is according to the EU index.

In another aspect, the disclosure provides a molecule comprising a non-protein agent conjugated to a FcRn binding fragment of an Fc region of an IgG molecule, wherein the FcRn binding fragment comprises: phenylalanine (F) at position 351; arginine (R), lysine (K), aspartate (D), glutamate (E), phenylalanine (F), tyrosine (Y), proline (P), glycine (G), leucine (L) or methionine (M) at position 354; arginine (R) at position 366; lysine (K) at position 395; arginine (R) at position 405; and glutamate (E) at position 407, wherein the amino acid numbering is according to the EU index.

In another aspect, the disclosure provides for nucleic acids encoding said fusion proteins or polypeptides or FcRn binding fragments conjugated to said molecules.

In another aspect, the disclosure provides vectors comprising said nucleic acids.

In another aspect, the disclosure provides for host cells comprising said vectors or nucleic acids.

In another aspect, the disclosure provides for methods of producing said fusion proteins, polypeptides of FcRn binding fragments for use in said molecules, by expressing said fusion proteins, polypeptides of FcRn binding fragments from said host cells and purifying therefrom.

In another aspect, the disclosure provides for the fusion proteins, polypeptides or molecules for use in therapy.

In another aspect, the disclosure provides for the use of the fusion proteins, polypeptides or molecules in the manufacture of a medicament for the treatment of a disease.

In another aspect, the disclosure provides methods of treatment comprising administering therapeutically effective amounts of the fusion proteins, polypeptides or molecules to a patient in need thereof.

### BRIEF DESCRIPTION OF DRAWINGS

FIG. 1A shows the sequence alignment of CH2 and CH3 domains in wild-type IgG4, MFc1, and MFc2 from a previous phage library campaign, T1 variant, which replaces the YTE mutations in the MFc1 CH2 domain with a CH3 mutation set, and final sequences of MFc3 and MFc4.

FIG. 1B shows the crystal structure of T1

FIG. 1C shows a zoomed view of T1 detailing a small set of hydrogen bonds formed in Thr350/Leu440 and Gln355/Glu356

FIG. 2A shows the sequence alignment of CH2 and CH3 domains in wild-type IgG4, T1, and T1-lib, a panel of point-mutation variants targeting residue S354.

FIG. 2B shows representative SEC-MALS analysis showing the T1 point mutants demonstrating a molecular weight of approximately 26-27 kDa with good homogeneity

FIG. 2C shows DSF comparisons among the T1 refinement mutants to identify S354E and S354D with moderately higher thermal stability

FIG. 3 shows the crystal structure of a new monomeric Fc, MFc3 in the aglycosylated form (N297D). Superposition of MFc3 (orange) with a previously resolved structure of MFc2 (or C4n) (light purple) shows that both maintained a similar monomeric Fc structure, and the S354E mutation did not cause any notable change in the Fc region structures. As designed, the glutamate side chain due to the S354E mutation protruded into any possible dimer interaction observed in T1

FIG. 4A shows the structural interrogation of MFc3 (orange) and FcRn (blue)/β2-macroglobulin (pink) complex

FIG. 4B shows the binding interface between MFc3 (orange) and FcRn (blue). Hydrogen bonds are indicated by dashed lines

FIG. 4C shows the MFc4 (green) and FcRn (blue)/β2-macroglobulin (pink) complex

FIG. 4D shows the binding interface between MFc4 (green) and FcRn (blue). Hydrogen bonds are indicated by dashed lines

FIG. 4E is a heat map showing the differential solvation energy AiG (kcal/mol) contribution from each MFc residue involved in the receptor binding interface. Higher positive values indicate stronger solvation effect from the monomeric Fc bound surface

FIG. 5A shows a cartoon of a monomeric Fc-based monovalent bispecific antibody

FIG. 5B is a SEC-MALS analysis of Fab-MFc1-scFv and Fab-MFc4-scFv showing measured molecular weight of approximately 100 kDa, with polydispersity of 1.001

FIG. 5C shows a concurrent binding analysis using biolayer interferometry demonstrating expected binding activity from both the Fab and scFv moieties to recombinant antigens

FIG. 6A is an in vivo mouse PK analysis of monomeric Fc-bispecific antibodies. hFcRn transgenic mouse serum clearance curves are plotted for Fab-MFc1-scFv, Fab-MFc4-scFv, Fab-MFc1, and IgG1 control, based on concurrent Fab and Fc region binding.

FIG. 6B shows the PK parameters determined by noncompartmental analysis with model 201. AUCINF=area under the concentration-time curve for the plasma-concentration versus time graph from time 0 to infinity; CL=clearance; Cmax=peak concentration; t½=terminal half-life.

### DETAILED DISCLOSURE

### Definitions

As used in this specification, the singular forms “a,” “an” and “the” specifically also encompass the plural forms of the terms to which they refer, unless the content clearly dictates otherwise.

The term “about” or “approximately” means an acceptable error for a particular value as determined by one of ordinary skill in the art, which depends in part on how the value is measured or determined. In certain instances, the term “about” or “approximately” means within 1, 2, 3, or 4 standard deviations. In certain instances, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series. In instances where “about” is used in conjunction with an amino acid position it means within 1, 2, 3, 4, 5 or 10 amino acids of the designated position.

“Amino acid deletion” or “deleted” refers to removing an amino acid residue present in a parent sequence. An amino acid can be deleted in a parent sequence, for example, through recombinant methods known in the art. Accordingly, references to a “deletion at position X” refers to the deletion of an amino acid present at position X. Deletion patterns can described according to the schema AX, wherein A is the single letter code corresponding to the amino acid naturally present at position X, and A is the deleted amino acid residue. Accordingly, L234 would refer to the deletion of the leucine amino acid (L) at position 234. Under such circumstances, residues 233 and 235 would then be encoded in sequence.

“Amino acid substitution” or “substitution” refers to replacing an amino acid residue present in a parent sequence with another amino acid residue. An amino acid can be substituted in a parent sequence, for example, via chemical peptide synthesis or through recombinant methods known in the art. Accordingly, references to a “substitution at position X” or “substitution at position X” refer to the substitution of an amino acid present at position X with an alternative amino acid residue. Substitution patterns can described according to the schema AXY, wherein A is the single letter code corresponding to the amino acid naturally present at position X, and A is the substituting amino acid residue. Accordingly, L234F would refer to the substitution of the leucine amino acid (L) at position 234 with a phenylalanine (F).

“Antibody” or is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An immunoglobulin, such as an immunoglobulin G (IgG) is an example of an antibody. “Class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, E, y, and 11, respectively.

“Antigen binding domain” refers to a molecule other than an intact antibody that comprises a portion of the intact antibody that binds to the antigen to which the intact antibody binds. Examples of antigen binding domains include, but are not limited to, Fv, Fab, Fab′, F(ab′)2, Fab′-SH, diabodies, triabodies, tetrabodies, linear antibodies, single-chain antibody molecules (e.g., scFv), and multispecific antibodies formed from antigen binding fragments. For a review of certain antigen binding domain, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

“EU Index” refers to the EU numbering index of Kabat et al. (Sequences of Proteins of Immunological Interest, 5^{th} ed., 1991 NIH Pub. No. 91-3242, which is incorporated by reference herein in its entirety). Amino acid residues of the FcRn binding fragments disclosed herein numbered according to this numbering system.

“Fab” refers to an antibody fragment comprising the VH-CH1 and VL-CL pairing. The term encompasses Fabs comprising non-canonical sequence variants such as amino acid substitutions, deletions or insertions within the Fab outside of sequence regions typically associated with high sequence variability. For example, Fab variants include Fabs comprising non-canonical amino acid or sequence changes in VH or VL framework regions or in the CH1 or CL domains. Such changes may include the presence of non-canonical cysteines or other derivatizable amino acids, which may be used to conjugate said Fab variants to heterologous moieties. Other such changes include the presence of non-canonical polypeptide linkers, which are polypeptide sequences that covalently bridge between two domains. For example, a Fab variant may comprise a linker polypeptide that covalently attaches the CH1 domain to the VL domain, or the CL domain to the VH domain, such that the Fab can be expressed as a single polypeptide chain.

“Fc region” or “Fc domain” refers to a C-terminal region of an immunoglobulin heavy chain that contains at least a portion of the constant region. It has no antigen binding activity but contains the carbohydrate moiety and the binding sites for complement and Fc receptors, including the FcRn receptor (see below). The Fc region contains the entire second constant domain CH2 (residues 231-340 of human IgG, according to the EU Index) and the third constant domain CH3 (residues 341-447). A reference sequence for a human IgG1 Fc region can be found via UniProtKB accession number P01857. A reference sequence for a human IgG4 Fc region can be found via UniProtKB accession number P01861.

“FcRn binding fragment” refers to a fragment of an Fc region that binds to the FcRn receptor. An FcRn-binding fragment can include portions of the heavy chain CH2-CH3 region or the hinge-CH2-CH3 region that are involved in binding to FcRn (see Roopenian et al., Nature Rev. Immunol. 7:715-725 (2007).

“FcRn receptor” or “FcRn” refers to an Fc receptor (“n” indicates neonatal) which is known to be involved in transfer of maternal IgGs to a fetus through the human or primate placenta, or yolk sac (rabbits) and to a neonate from the colostrum through the small intestine. It is also known that FcRn is involved in the maintenance of constant serum IgG levels by binding the IgG molecules and recycling them into the serum. The binding of FcRn to naturally occurring IgG1, IgG2, and IgG4 molecules is strictly pH-dependent with optimum binding at pH 6. IgG3 has a known variation at position 435 (i.e., human IgG has R435 instead of H435 found in human IgG1, IgG2 and IgG4), which may result in reduced binding at pH 6. FcRn comprises a heterodimer of two polypeptides, whose molecular weights are approximately 50 kD and 15 kD, respectively. The extracellular domains of the 50 kD polypeptide are related to major histocompatibility complex (MHC) class I α-chains and the 15 kD polypeptide was shown to be the non-polymorphic β₂-microglobulin (β₂-m). In addition to placenta and neonatal intestine, FcRn is also expressed in various tissues across species as well as various types of endothelial cell lines. It is also expressed in human adult vascular endothelium, muscle vasculature and hepatic sinusoids and it is suggested that the endothelial cells may be most responsible for the maintenance of serum IgG levels in humans and mice.

“Fusion protein” refers to a chimeric polypeptide which comprising a first domain linked to a second domain with which it is not naturally linked in nature. Fusion proteins may comprise more than two domains.

“Hinge-Fc region”, “Fc-hinge region,” “hinge-Fc domain” or “Fc-hinge domain”, as used herein are used interchangeably and refer to a region of an IgG molecule consisting of the Fc region (residues 231-447, numbered according to the EU index) and a hinge region (residues 216-230, numbered according to the EU index) extending from the N-terminus of the Fc region.

“Host cell” refers to the particular subject cell transfected with a nucleic acid molecule or infected with phagemid or bacteriophage and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

“Linked,” “fused,” or “fusion” are used interchangeably. These terms refer to the joining together of two or more elements or components, by whatever means, including chemical conjugation or recombinant means.

“Polynucleotide,” or “nucleic acid,” refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs.

“ScFv” refers to an antibody fragment comprising a VH/VL domain pairing of an antibody. An scFv comprises a polypeptide linker between the VH and VL domain. An scFv may also comprise non-canonical amino acid sequence variants, such as engineered cysteines. An scFv may comprise a pair of engineered cysteines for intra-domain disulfide bond formation.

“Subject” refers to an animal, human or non-human, to whom treatment according to the methods of the present invention is provided. Veterinary and nonveterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs. The preferred subject is a human.

“Therapeutically effective amount” refers to an amount of the fusion protein, polypeptide, molecule, or pharmaceutical compositions thereof, effective to “treat” a disease or disorder in a subject or mammal.

“Treating” or “treatment” or “to treat” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for a disease or condition, for example, cancer, according to the methods of the present disclosure if the patient shows, e.g., total, partial, or transient remission of the disease or condition, for example, a certain type of cancer.

“Vector” refers to a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

### FcRn Binding Fragments

Fc regions are non-antigen binding components of antibodies that facilitate a range of antibody-mediated functionalities. One particular function is to engage with FcRn receptors to facilitate antibody recycling to modulate antibody half-life. Higher affinity binding at endosomal pH between Fc regions and FcRn facilitates trafficking of antibodies from acidic endosomes to rescue an antibody from lysosomal degradation.

The present disclosure relates to the development of a FcRn binding fragment stabilised in a monomeric form that can be used to prolong the half-life of a range of therapeutic modalities. The enhancement is achieved by enabling the FcRn-recycling properties of the Fc region to be achieved in the monomeric form. The FcRn binding fragments can be used to create fusion proteins to expand the repertoire of therapeutic modalities that benefit from FcRn recycling.

Accordingly, the disclosure provides a FcRn binding fragment of an Fc region of an IgG molecule. The FcRn binding fragment comprises F at position 351, R, K, D, E, F, Y, P, G, L or M at position 354, R at position 366, K at position 395, R at position 405, and E at position 407. Amino acid numbering is according to the EU index.

The examples show that these amino acid substitutions relative to the wild-type (wt) sequence of an Fc region of an IgG molecule improve monomer stability of the FcRn binding fragment. Fc regions in wt antibodies typically exist in a dimerised form. Improved monomer stability is advantageous where the FcRn binding fragment is to be utilised in a fusion protein in which it is desirable for the therapeutic protein to be monomeric (for example, in gene therapy applications or for delivery by inhalation, where packing size limitations or aerosolization properties preclude the administration of standard antibodies via these routes). The expression and purification of the fusion proteins, polypeptides or FcRn binding fragments for conjugation to non-protein agents may also improve yield in large scale manufacturing compared to isolating therapeutic proteins with a quaternary structure.

Generally, the amino acids at each of the designated positions in the sequence are non-canonical, meaning they are not typically found at the designated positions in wild-type Fc regions, particularly wild-type human Fc regions. The amino acid modifications (e.g., substitutions, deletions or insertions) may be engineered into the sequence using standard genetic engineering techniques well-known to the skilled person.

Accordingly, the disclosure provides a FcRn binding fragment of an Fc region of an IgG molecule. The FcRn binding fragment comprises the following amino acid substitutions: F at position 351, R, K, D, E, F, Y, P, G, L or M at position 354, R at position 366, K at position 395, R at position 405, and E at position 407. Amino acid numbering is according to the EU index.

Unless otherwise stated herein, reference to a particular amino acid at a particular position in the Fc region (numbered according to the EU index) means that the amino acid is a substitution at that particular position compared to the native sequence.

In some instances, the FcRn binding fragment further comprises R, K, D or E at position 354, optionally D or E, wherein the numbering is according to the EU index. The examples show that S354 assists dimerization of FcRn binding fragments at high concentrations. The formation of higher-order species at high concentrations may be undesirable for particular therapeutics which are administered as high concentration solutions via subcutaneous injection. Certain therapies may require relatively high (e.g., in excess of 300 mg) amounts to be administered to achieve therapeutic efficacy. High amounts may require high concentration formulations to reduce the injection volume. Greater injection volumes are generally undesirable for a patient. The formation of higher-order species at high concentrations may increase viscosity, which could be problematic when the drug product is to be administered from a pressurised device, such as a pre-filled syringe. Furthermore, greater monomer purity at high concentrations may increase production yield if higher-order species are lost during the manufacturing process, such as during filtration or fractionation. In some instances, the FcRn binding fragment further comprises E at position 354.

In some instance, the FcRn binding fragment comprises from about amino acid residues from about amino acid residue 216 to about amino acid residue 447 of an IgG molecule, wherein the numbering is according to the EU index. Residues 216-447 comprise the hinge-Fc region (residues 216-230, numbered according to the EU index) and the Fc region (residues 231-447, numbered according to the EU index).

In some instance, the FcRn binding fragments comprise from about amino acid residues from about amino acid residue 231 to about amino acid residue 447 of an IgG molecule, wherein the numbering is according to the EU index.

In certain instances, the FcRn binding fragment is derived from the IgG4 subclass of IgGs, but may also be any other IgG subclasses of given animals. For example, in humans, the IgG class includes IgG1, IgG2, IgG3, and IgG4. In certain instances, the FcRn binding fragment is derived from an IgG4 Fc region.

In some instances, the FcRn binding fragment comprises: F at position 351, E at position 354, R at position 366, K at position 395, R at position 405, and E at position 407, wherein amino acid numbering is according to the EU Index.

In some instances, the FcRn binding fragment may comprises a conservative amino acid substitution, with respect to each of the these amino acids at each of these amino acid positions: F at position 351, E at position 354, R at position 366, K at position 395, R at position 405, and E at position 407, wherein amino acid numbering is according to the EU Index.

Exemplary conservative amino acid substitutions for each of these amino acids are as follows:

| Amino Acid | Conservative Substitution |
|---|---|
| F | M, Y, W, I, L, V |
| E | Q, D, K, N, H, R |
| R | H, L, E, Q |
| K | R, E, Q, H |

A conservative substitution is an amino acid replacement in a protein that changes a given amino acid to a different amino acid with similar biochemical properties (e.g., charge, hydrophobicity and size). The skilled person may therefore expect a conservative amino acid substitution to generate similar benefits with respect to monomer formation and half-life modification compared to the most exemplary FcRn binding fragment described herein. The skilled person can generate FcRn binding fragments with conservative amino acid substitutions at one or more of positions 351, 354, 366, 395, 405 and 407 (numbered according to the EU index) and test whether these variants have similar properties (e.g., monomer stability, FcRn binding potency, half-life characteristics) to the preferred FcRn binding fragments disclosed herein, by carrying out the experiments described in the examples.

In some instances, the FcRn binding fragment comprises the amino acid sequence set forth in SEQ ID NO:1. In some instances, the FcRn binding fragment has the amino acid sequence set forth in SEQ ID NO: 1. In some instances, the FcRn binding fragment consists of the amino acid sequence set forth in SEQ ID NO: 1.

In some instances, the FcRn binding fragment comprises half-life extension mutations (e.g., amino acid insertions, deletions or substitutions).

In some instances, the FcRn binding fragment has an equilibrium dissociation constant (K_{D}) for human FcRn of less than 300 nM. The examples show that human IgG1 Fc region binds to FcRn at pH 6 with a K_{D} of approximately 300 nM. By contrast, the engineered FcRn binding fragments can bind to human FcRn with a substantially improved K_{D} at pH 6.0. Tighter binding at lower pH means that recycling propensity from endosomes may be improved compared to FcRn binding fragments comprising the native sequence.

The K_{D} can be measured by any number of techniques well-known to the skilled person, including the technique outlined in the examples. For example, binding measurements of FcRn binding fragments to purified recombinant human FcRn may be carried out by biolayer interferometry. Biolayer interferometry may be carried out using an Octet384 instrument (ForteBio, Menlo Park, CA). For example, biotinylated FcRn at 1 μg/mL in PBS buffer (pH 7.4) or 100 mM MES buffer (pH 6.0), with 3 mg/mL bovine serum albumin, 0.05% (vol/vol) and Tween 20 (1× Kinetics Buffer; ForteBio), could be captured on streptavidin biosensors (ForteBio). The loaded biosensors can then be washed with assay buffer to remove any unbound protein, followed by association and dissociation measurements with serial dilutions of the different Fc variants or Fc fusion constructs at the desired pH. Octet software (version 7.2) can then used to calculate kinetic parameters (kₒₙ and k_{off}) and apparent K_{D} from a nonlinear fit based on the 1:1 binding kinetic model of the data, with the equation

$$( A + B {↔}_{{k}_{off}}^{{k}_{on}} AB ) .$$

In some instances, the FcRn binding fragment has an equilibrium dissociation constant (K_{D}) for human FcRn of at least 300 nM.

In some instances, the FcRn binding fragment binds to human FcRn with a K_{D} of from about 1 nM and about 300 nM at pH 6 (e.g., between about 1 nM and about 250 nM, between about 1 nM and about 240 nM, between about 1 nM and about 230 nM, between about 1 nM and about 200 nM, between about 1 nM and about 180 nM, between about 1 nM and about 160 nM, between about 1 nM and about 140 nM, between about 1 nM and about 120 nM, between about 1 nM and about 100 nM, between about 1 nM and about 80 nM, between about 1 nM and about 60 nM, between about 1 nM and about 40 nM, between about 1 nM and about 20 nM, or between about 1 nM and about 100 nM). In some instances, the FcRn binding fragment binds to human FcRn with a K_{D} of from about 1 nM and about 10 nM at pH 6. In some instances, the FcRn binding fragment binds to human FcRn with a K_{D} of about 1 nM, about 2 nM, about 3 nM, about 4 nM, about 5 nM, about 6 nM, about 7 nM, about 8 nM, about 9 nM or about 10 nM. In some instances, the FcRn binding fragment binds to human FcRn with a K_{D} of about 5 nM at pH 6.0. In some instances, the FcRn binding fragment binds to human FcRn with a K_{D} of 5 nM at pH 6.0.

In some instances, K_{D} values may be determined by biolayer interferometry, for example, as described above and in the examples.

### Antigen Binding Domains

In certain instances, the disclosure provides fusion proteins comprising at least one antigen-binding domain covalently linked to a FcRn binding fragment disclosed herein.

In some instances, the fusion protein or polypeptide comprises multiple antigen binding domains.

In some instances, the fusion protein or polypeptide comprises 1, 2, 3, 4 or 5 antigen binding domains.

In some instances, the fusion protein or polypeptide comprises two antigen binding domains.

In some instances, a first antigen binding domain is N-terminal to the FcRn binding fragment.

In some instances, a second antigen binding domain is C-terminal to the FcRn binding fragment.

In some instances, each antigen binding domain is N-terminal to the FcRn binding fragment.

In some instances, each antigen binding domain is C-terminal to the FcRn binding fragment.

In some instances, each antigen binding domain binds specifically to a different antigen.

In some instances, each antigen binding domain is independently selected from Fv, Fab, Fab′, F(ab′)2, Fab′-SH, diabody, triabody, tetrabody, linear antibody or scFv.

In some instances, an antigen binding domain is a Fab.

In some instances, an antigen binding domain is an scFv.

In some instances, the fusion protein or polypeptide comprises a first and second antigen binding domains that are Fabs.

In some instances, the fusion protein or polypeptide comprises a first and second antigen binding domains that are scFvs.

In some instances, the fusion protein or polypeptide comprises a first antigen binding domain that is a Fab and a second antigen binding domain that is a scFv.

The examples show that the FcRn binding fragment disclosed herein can be used in the generation of bispecific molecules

### Fc Region Variants

In certain instances, one or more amino acid modifications may be introduced into the Fc region, thereby generating an Fc region variant. The Fc region variant may then be incorporated into the FcRn binding fragments disclosed herein. The Fc region variant may comprise a human Fc region sequence (e.g., a human IgG1, IgG2, IgG3 or IgG4 Fc region) comprising an amino acid modification (e.g. a substitution) at one or more amino acid positions.

In certain instances, the FcRn binding fragment possesses some but not all effector functions, which makes it a desirable candidate for applications in which the half-life of the FcRn binding fragment in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g, in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Intl. Immunol. 18(12): 1759-1769 (2006)).

In some instances, one or more amino acid modifications may be introduced into the Fc region in order to increase binding to FcRn. In some instances, the FcRn binding fragment comprises the following three mutations, numbered according to the EU index: M252Y, S254T, and T256E (the “YTE mutation”) (U.S. Pat. No. 8,697,650; see also Dall'Acqua et al., Journal of Biological Chemistry 281(33):23514-23524 (2006).

In certain instances, the YTE mutation increases the FcRn binding fragment's serum half-life compared to the native (i.e., non-YTE mutant) FcRn binding fragment. In some instances, the YTE mutation increases the serum half-life of the FcRn binding fragment by 2-fold compared to the native (i.e., non-YTE mutant) FcRn binding fragment. In some instances, the YTE mutation increases the serum half-life of the FcRn binding fragment by 3-fold compared to the native (i.e., non-YTE mutant) FcRn binding fragment. In some instances, the YTE mutation increases the serum half-life of the FcRn binding fragment by 4-fold compared to the native (i.e., non-YTE mutant) FcRn binding fragment. In some instances, the YTE mutation increases the serum half-life of the FcRn binding fragment by at least 5-fold compared to the native (i.e., non-YTE mutant) FcRn binding fragment. In some instances, the YTE mutation increases the serum half-life of the FcRn binding fragment by at least 10-fold compared to the native (i.e., non-YTE mutant) FcRn binding fragment. See, e.g., U.S. Pat. No. 8,697,650; see also Dall'Acqua et al., Journal of Biological Chemistry 281(33):23514-23524 (2006).

In some instances, the FcRn binding fragment is mutated to reduce effector function. In some instances, FcRn binding fragments with reduced effector function include those with substitution of one or more of Fc region residues 238, 265, 269, 270, 297, 327 and 329 numbered according to the EU index (U.S. Pat. No. 6,737,056). Such mutated FcRn binding fragment include those with substitutions at two or more of amino acid positions 265, 269, 270, 297 and 327 numbered according to the EU index, including the so-called “DANA” Fc region mutant with substitution of residues 265 and 297 to alanine, numbered according to the EU index (i.e., D265A and N297A according to EU numbering) (U.S. Pat. No. 7,332,581). In certain instances the FcRn binding fragment comprises the following two amino acid substitutions: D265A and N297A. In certain instances the FcRn binding fragment consists of the following two amino acid substitutions: D265A and N297A.

In certain instances, the proline at position 329 (numbered according to the EU index) (P329) is substituted with glycine or arginine or an amino acid residue large enough to destroy the proline sandwich within the Fc/Fcγ receptor interface, that is formed between the P329 of the Fc and tryptophane residues W87 and WI10 of FcgRIII (Sondermann et al.: Nature 406, 267-273 (20 Jul. 2000)). In a further instances, at least one further amino acid substitution in the FcRn binding fragment is S228P, E233P, L234A, L235A, L235E, N297A, N297D, or P331S and still in another instance, said at least one further amino acid substitution is L234A and L235A, of the Fc region of the human IgG1 or S228P and L235E, of the Fc region of human IgG4, all numbered according to the EU index (U.S. Pat. No. 8,969,526).

In some instances, the FcRn binding fragment comprises one or more substitutions described in US2005/0014934A1, which improve binding of the Fc region to FcRn. Such FcRn binding fragments include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826) numbered according to the EU index. See also Duncan & Winter, Nature 322:738-40 (1988); U.S. Pat. Nos. 5,648,260; 5,624,821; and WO 94/29351 concerning other examples of Fc region variants.

In some instances, the FcRn binding fragment comprises His435 loop mutations described in WO2015/175874, which is incorporated herein by reference in its entirety, that modulate binding to FcRn. The examples show that His435 loop mutations enhance FcRn binding fragment serum half-life compared to FcRn binding fragments comprising the native amino acid residues at the positions 432-437, numbered according to the EU index.

In some instances, the Fc region comprises the following substitutions: C at position 432; H, R, P, T, K, S, A, M or N at residue 433; Y, N, R, W, H, F, S, M or T at residue 434; H at residue 435; L, Y, F, R, I, K, M, V, H, S or T at residue 436; and C at residue 437. In some instances, the in vivo half-life of the modified FcRn binding fragment is increased native (i.e., non-YTE mutant) FcRn binding fragment. Increasing the in vivo half-life of bioactive molecules has many benefits including reducing the amount and/or frequency of dosing of these molecules, for example, in vaccines, passive immunotherapy and other therapeutic and prophylactic methods

In some instances, the FcRn binding fragment variant comprises the following substitutions: C at position 432; S, H, R, P, T, K, A, M or N at residue 433; Y, R, W, H or F at residue 434; H at residue 435; L, R, I, K, M, V or H at residue 436; and C at residue 437, wherein numbering is according to the EU index. In some instances, the FcRn binding fragment comprises: C at position 432; S at position 433, W or Y at position 434; H at position 435; L at positions 436 and C at position 437, wherein numbering is according to the EU index. In some instances, the FcRn binding fragment variant comprises C at position 432; S at position 433, Y at position 434; H at position 435; L at position 436 and C at position 437, wherein numbering is according to the EU index. WO2015/175874 and the examples of the present disclosure show that this particular combination of mutations increases pH-dependent binding of an Fc region to FcRn, increasing pH-dependent FcRn-mediated recycling, thereby improving half-life.

In some instances, the FcRn binding fragment variant comprises deletion of the amino acid at position 438 numbered according to the EU index. The examples surprisingly show that deleting this amino acid from the sequence generates FcRn binding fragments with at least comparable half-life extension properties without comprising the developability profile. In some instances, the FcRn binding fragment comprises deletion of Q438, wherein the numbering is according to the EU index.

### Methods of Treatment

The fusion protein, polypeptide or molecule disclosed herein may be used in a method of therapy, for example, in a method of treating cancer. Also provided is a method of treatment, comprising administering to a subject in need of treatment a therapeutically-effective amount of a fusion protein, polypeptide or molecule disclosed herein. The actual amount administered, and rate and time-course of administration, will depend on the nature and severity of what is being treated. Prescription of treatment, e.g. decisions on dosage, is within the responsibility of general practitioners and other medical doctors.

### Nucleic Acids

The present disclosure provides polynucleotides comprising nucleic acid sequences that encode a fusion protein, polypeptide or FcRn binding fragment of a molecule disclosed herein. These polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand. In certain instances the DNA is a cDNA that is used to produce a non-naturally-occurring recombinant fusion protein, polypeptide or FcRn binding fragment of a molecule.

In certain instances, the polynucleotides are isolated. In certain instances, the polynucleotides are substantially pure. In certain instances the polynucleotides comprise the coding sequence for the mature polypeptide fused in the same reading frame to a polynucleotide (either natural or heterologous) which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. In certain instances, the polynucleotides are altered to optimize codon usage for a certain host cell.

In certain instances, the polynucleotides comprise the coding sequence for the fusion protein, polypeptide or FcRn binding fragment of a molecule fused in the same reading frame to a heterologous marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

The polynucleotides can contain alterations in the coding regions, non-coding regions, or both. In some embodiments, these polynucleotide variants contain alterations that produce silent substitutions, additions, or deletions, but do not alter the properties or activities of the encoded polypeptide. In some embodiments, the polynucleotide variants are produced by silent substitutions due to the degeneracy of the genetic code. Polynucleotide variants can also be produced for a variety of reasons, e.g., to optimize codon expression for a particular host (change codons in the human mRNA to those preferred by a bacterial host such as E. coli).

Vectors and cells comprising the polynucleotides described herein are also provided. Once assembled (by synthesis, site-directed mutagenesis or another method), the polynucleotide sequences encoding a particular isolated polypeptide of interest can be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain instances, recombinant expression vectors are used to amplify and express DNA encoding the fusion protein, polypeptide or FcRn binding fragment of a molecule disclosed herein. Recombinant expression vectors are replicable DNA constructs which have synthetic or cDNA-derived DNA fragments encoding, for example, a polypeptide chain of a fusion protein, polypeptide or FcRn binding fragment of a molecule, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

Suitable host cells for expression of fusion proteins, polypeptides or FcRn binding fragments of molecules disclosed herein, include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Publ. No. 2008/0187954, U.S. Pat. Nos. 6,413,746 and 6,660,501, and Int'l Pat. Publ. No. WO 04009823, each of which is hereby incorporated by reference in its entirety.

Fusion proteins, polypeptides or FcRn binding fragments of molecules produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

In another aspect, the disclosure provides a method of making a fusion protein, polypeptide or FcRn binding fragment as defined herein. The method comprises (i) mutagenizing a nucleic acid sequence encoding an FcRn binding fragment by replacing the codons at amino acid position 351, 354, 366, 395, 405 and 407 with codons that encode amino acids at each position as described herein. The amino acid position numbering is according to the EU index. The method further comprises expressing the mutagenized nucleic acid sequence; and isolating the expressed fusion protein, polypeptide or FcRn binding fragment.

In some instances, the method comprises the further step of reacting the FcRn binding fragment with a non-protein agent to form a molecule, as disclosed herein.

### Pharmaceutical Compositions

The present disclosure extends to compositions comprising a fusion protein, polypeptide or molecule described herein, in particular a pharmaceutical composition (or diagnostic composition) comprising or fusion protein, polypeptide or molecule of the present disclosure and pharmaceutical excipient, diluent or carrier.

The composition will usually be supplied as part of a sterile, pharmaceutical composition that will normally include a pharmaceutically acceptable carrier. A pharmaceutical composition of the present invention may additionally comprise a pharmaceutically-acceptable adjuvant in the context of vaccine formulation.

The disclosure also extends to processes of preparing said compositions, for example preparation of a pharmaceutical or diagnostic composition comprising adding and mixing a molecule of the present disclosure, such as hydrolysed molecule of the disclosure of the present invention together with one or more of a pharmaceutically acceptable excipient, diluent or carrier.

The fusion protein, polypeptide or molecule of the disclosure may be the sole active ingredient in the pharmaceutical or diagnostic composition or may be accompanied by other active ingredients.

The pharmaceutical compositions suitably comprise a therapeutically effective amount of a fusion protein, polypeptide or molecule according to the disclosure. The therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

Compositions may be administered individually to a patient or may be administered in combination (e.g. simultaneously, sequentially or separately) with other agents, drugs or hormones.

The pharmaceutically acceptable carrier should not itself induce the production of antibodies harmful to the individual receiving the composition and should not be toxic.

Pharmaceutically acceptable carriers in therapeutic compositions may additionally contain liquids such as water. Auxiliary substances, such as wetting or emulsifying agents or pH buffering substances, may be present in such compositions. Such carriers enable the pharmaceutical compositions to be formulated as tablets, pills, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion and it may contain formulatory agents, such as suspending, preservative, stabilising and/or dispersing agents. Alternatively, the molecule of the disclosure may be in dry form, for reconstitution before use with an appropriate sterile liquid.

Suitably in formulations according to the present disclosure, the pH of the final formulation is not similar to the value of the isoelectric point of the fusion protein, polypeptide or molecule, for example if the pH of the formulation is 7 then a pI of from 8-9 or above may be appropriate.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

A thorough discussion of pharmaceutically acceptable carriers is available in Remington's Pharmaceutical Sciences (Mack Publishing Company, N.J. 1991).

### EXAMPLES

### Example 1

The field of drug discovery has made remarkable progress in delivering paradigm-shifting antibody and antibody-derivative therapeutics along with breakthroughs in both disease biology and antibody technology.^{1,2} The synergy between research advances in immuno-oncology and the development of novel bispecific and multispecific targeting platforms has produced many promising possibilities for driving the latest success in cancer treatment.³⁻⁷ The importance of minimizing toxicity while achieving clinical efficacy has highlighted the need to fine-tune targeting valency from the default bivalent format of immunoglobulin Gs (IgGs) or fragment crystallizable (Fc) fusion proteins. Increasing evidence indicates that bispecific and multi-specific formats with monovalent targeting arms are necessary for reducing nonspecific cell killing, cytokine release, and undesired receptor crosslinking and improving receptor agonism and transport.⁸⁻¹¹ T-cell engagers or natural-killer (NK) cell engagers, such as bispecific T-cell engagers (BiTe), dual-affinity re-targeting proteins (DART), bispecific killer-cell engagers (BiKE), and trispecific killer-cell engagers (TriKE), have shown clinical promise with monovalent bispecific and multispecific targeting but suffer from short half-lives. Heterodimeric Fc engineering has made it possible to extend the half-lives of these engagers via the development of technologies such as knob-in-hole, CrossmAb, and DuetmAb, which can direct correct chain pairing for bispecific and multispecific targeting.¹¹⁻¹³

The successful generation of a conformationally stable, monomeric Fc antibody fragment with a “tunable” serum half-life could open new possibilities for antibody and Fc fusion therapeutics. Of the more than 180 therapeutic proteins that have received approval from the U.S. Food and Drug Administration, many have the potential to be fitted into active, longer-lasting fusion proteins.¹⁴⁻¹⁸ Immune-cell engagers, antibody-drug conjugates, immunocytokine fusions, and other therapeutic proteins could be tailor-designed to provide monovalent targeting in both monospecific and multispecific formats for enhanced activity and reduced toxicity. Past efforts to engineer a monomeric Fc, defined as one set of CH2 and CH3 domains, have proved challenging due to the extensive interactions to be disrupted in the CH3-CH3 dimer interface. At high concentrations, monomer-dimer equilibrium has been observed in many mutants.^{19,20} Among the engineered monomeric Fc modalities, only two molecules have been reported to have crystal structures with demonstrable homogeneity and stability.^{13,21} One of these is a monomer that is stabilized by the addition of a glycosylation site that blocks the CH3-CH3 interaction.²¹ The other, which was generated by our group, is a monomeric Fc derived from an IgG4 phage library that was rationally designed on the basis of previous findings.¹³

To broaden the application of the monomeric Fc platform for the next wave of protein therapeutics, we set out to address three important, interconnected aspects of this endeavor: (1) a tunable serum half-life, (2) versatile construction of monovalent bispecific molecules, and (3) facile structural interrogation of Fc neonatal receptor (FcRn) interaction with Fc variants. The pH-dependent Fc-FcRn interaction is a key contributor to the prolonged serum half-life of antibodies and their derivatives. FcRn harnesses antibody molecules and carries them through the acidic endosomal vesicles, protects them from lysosomal degradation, and releases them outside the cells due to weak binding at neutral pH^{22,23} Monomeric Fc variants are expected and observed to have reduced apparent binding to FcRn with loss of dimer avidity.^{13,21} Previously, we circumvented the loss of FcRn binding by building the YTE (N252Y/S254T/T256E) mutations into the phage library template design. The resulting monomeric Fc molecule yielded greater than 10-fold improvement in FcRn binding affinity compared with its counterpart without YTE.^{13,24}

In this work, we report a structure-guided approach to engineer a monomeric Fc molecule that is adaptable for half-life extension modifications beyond those achieved with the previously built-in YTE mutations. This is the first proof-of-concept monomeric bispecific molecular design demonstrating that it is possible to achieve significant improvement in in vivo serum half-life with only one copy of the CH2-CH3 domain. The co-crystal structures of these monomeric Fc molecules with FcRn revealed details of the interface that could serve as a basis for building other half-life extensions.

### Results

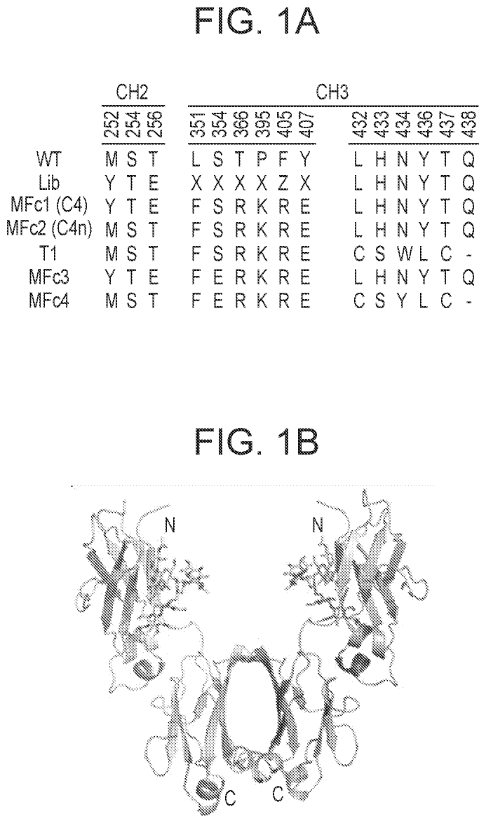

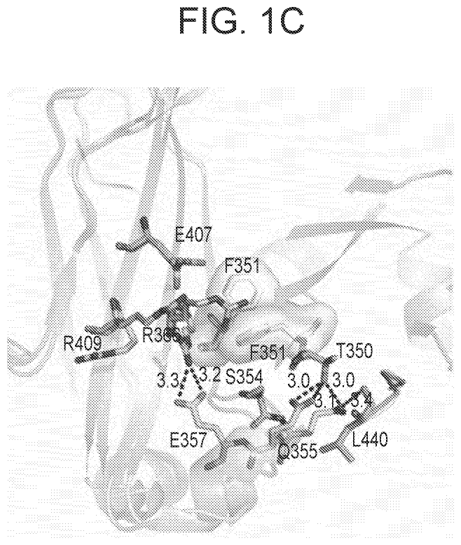

Disruption of CH3-CH3 interface based on structural insights. In previous work, we generated a monomeric Fc variant, C4 (now renamed MFc1), from a rationally designed IgG4 phage library containing a set of mutations in the CH3 domain to fully stabilize the disruption of the Fc dimer interface (FIG. 1a). To extend the compatibility of these dimer-disrupting mutations with half-life extension mutations other than YTE, we initiated our effort with a test variant, T1, by replacing the YTE mutations in the CH2 domain of the monomeric Fc sequence with a new set of half-life modification mutations in CH3 around residues 432-438. This mutation set was chosen because it fit the following two criteria. First, the new mutation set could help us to explore further methods of half-life enhancement, based on the findings from a previous phage library campaign showing that it could achieve significant improvement in FcRn binding over those of the YTE mutation.²⁵ Second, the extensive nature of this set of mutations would be a “stress test” on the ability of the monomeric Fc to sustain disruptions in the Fc dimerization interface.

We solved the crystal structure for T1 and analyzed it alongside that of the dimeric IgG4 Fc and the previously solved structure of the monomeric Fc, MFc2 (or C4n, with no YTE mutations; PDB ID: 5HVW))¹³ The CH3 domains of the MFc2 and IgG4 Fc (PDB ID: 4C54) were superimposed with T1 with a root-mean-square deviation (RMSD) of ˜0.7 and 0.5 Å over the Ca atoms, respectively (when excluding the last 11 residues that form an artificial chain swap in the MFc2 structure). Despite the high degree of similarity of CH3 domains, T1 unexpectedly showed a formation of homodimers (FIG. 1b). The dimer interface, however, differed dramatically from that of wild-type IgG4. A small set of hydrogen bonds involving amino acids Thr350/Leu440 and Gln355/Glu356 were formed between the chains, indicating a possible breach of the monomeric Fc formation (FIG. 1c). Upon inspection of the newly formed T1 dimer interface superimposed with MFc2, we observed that one of the mutations in MFc2, namely, Arg366, had shifted position in its side chain. This shift allowed for the Phe351 from each chain to “reach in” and form a hydrophobic stacking interaction. Additionally, the Arg366 mutation established few new hydrogen bonds that stabilized the dimer (FIG. 1c). From a close examination of the structure, we reasoned that the Arg366 shift became possible partly due to the existence of a space above the Ser354 side chain. Although analytical methods showed a relatively well-behaving T1 protein, the crystal structure suggests that despite the extensive disruption in dimer interface, there remained a possibility that at high protein concentrations the engineered Fc could be engaged in dimer formation.

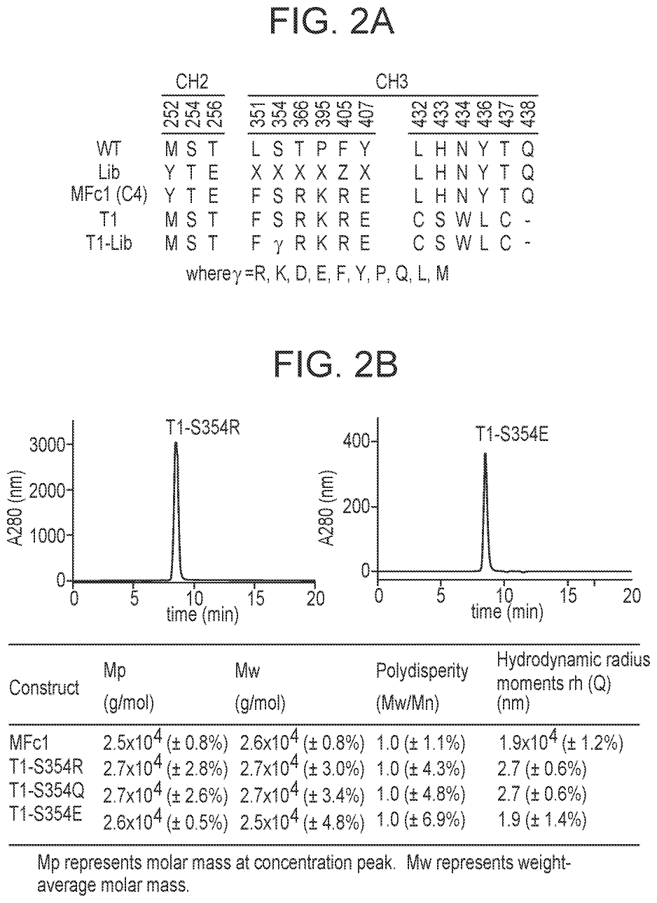

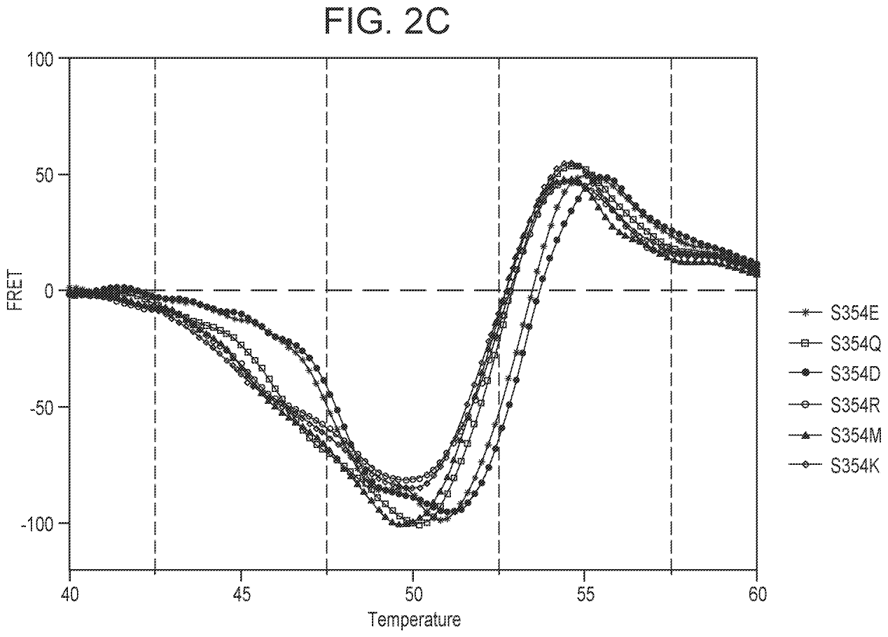

Structure-based engineering and characterization of binding and biophysical properties. The newly formed interaction observed in the T1 crystal structure created an opportunity to build a more adaptable monomeric Fc molecule. From the structural inspection of the T1 dimer interface (FIG. 1c), we posited that replacing serine at position 354 with an amino acid with a bulkier side chain might prevent the Arg366 side chain from clearing the way for a hydrophobic interaction involving Phe351. This substitution could also disrupt any resulting hydrogen bond formation. Interestingly, Ser354 had been selected as one of the interfacial positions to mutate in the original phage library template, even though our first monomeric Fc construct, MFc1, had produced a mutation at every targeted position except Ser354 (FIG. 1a).¹³ We therefore designed a small panel of refinement mutations at position 354 to investigate whether introducing a larger side chain or electrostatic repulsion would completely stabilize the monomer formation. T1 variants containing substitutions at position 354 (T1-lib) with charged residues (R, K, D, E) and bulky polar and nonpolar residues (F, Y, P, Q, L, M) were constructed (FIG. 2a) and subsequently purified by protein A affinity chromatography. SEC-MALS analysis revealed that nearly all the T1-lib refinement variants were monomeric (FIG. 2b).

To choose the most stable monomer among the T1-lib variants, we used differential scanning fluorimetry (DSF), which has been established as an orthogonal screening tool to assess thermal unfolding as a function of hydrophobic (Tₕ) residue exposure.^{32,33} Thermal unfolding in the T1-lib variants was monitored and changes were observed for the transition temperature for hydrophobic exposure, Tₕ. Notably, the types of the amino acid substitutions had an impact on the Tₕ ranking, as the acidic residues (Glu and Asp) generated a higher transition temperature of up to 3° C. than basic residues (Arg and Lys) (FIG. 2c).

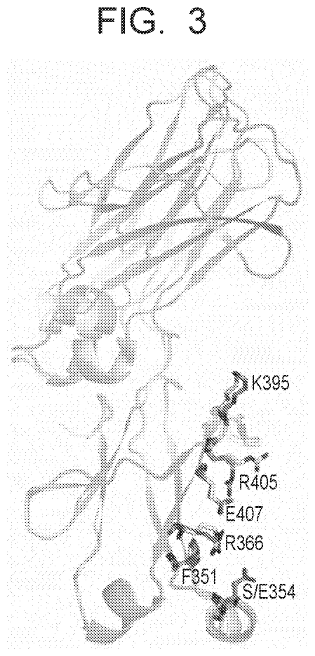

Structures and properties of monomeric Fc variants with S354E (MFc3 and MFc4). Based on SEC-MALS and DSF results, the S354E mutation was selected to be explored for general applicability of the MFc platform. First, to confirm its compatibility with our original monomeric Fc construct, we generated MFc3, the S354E point mutant of MFc1, along with its aglycosylated variant (N297D), for crystal structural confirmation (FIG. 1a). Crystals of the aglycosylated MFc3 protein grew readily and diffracted to 2.4 Å. The solved structure demonstrated that the MFc3 molecule maintained the monomeric state at high protein concentration (FIG. 3). Superposition with the previously published monomeric Fc structure for MFc2 showed that the S354E mutation did not cause any major change in the structure of the CH2 or CH3 domains of Fc.¹³ Aside from a minor change of the R405 side chain position, all the other original set of dimer-disruptive mutations in MFc1 aligned nearly perfectly with each other. Importantly, this crystal structure demonstrated that, as designed, the glutamic acid side chain substitution at position 354 indeed protruded out and disrupted the kind of interaction we observed in T1.

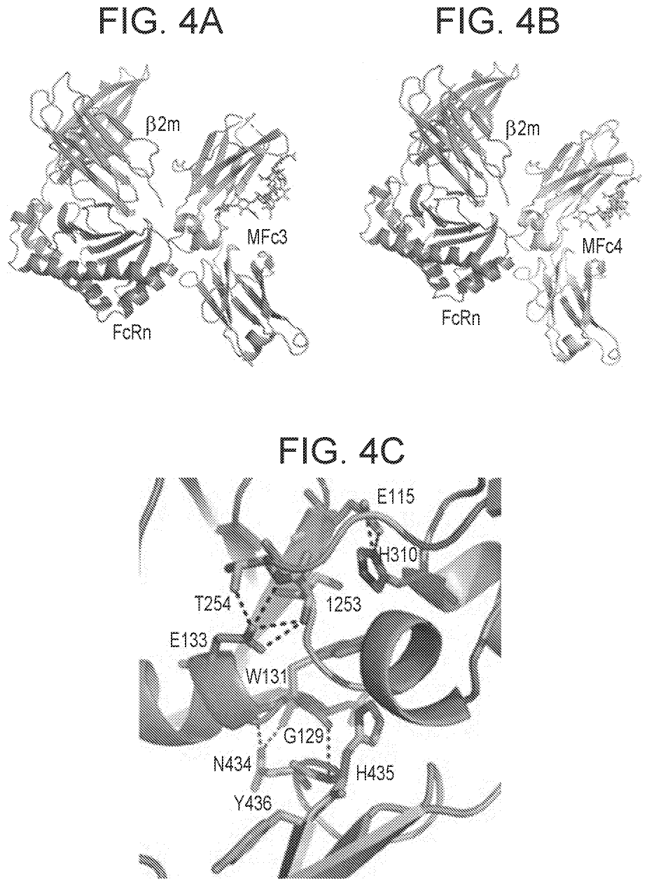

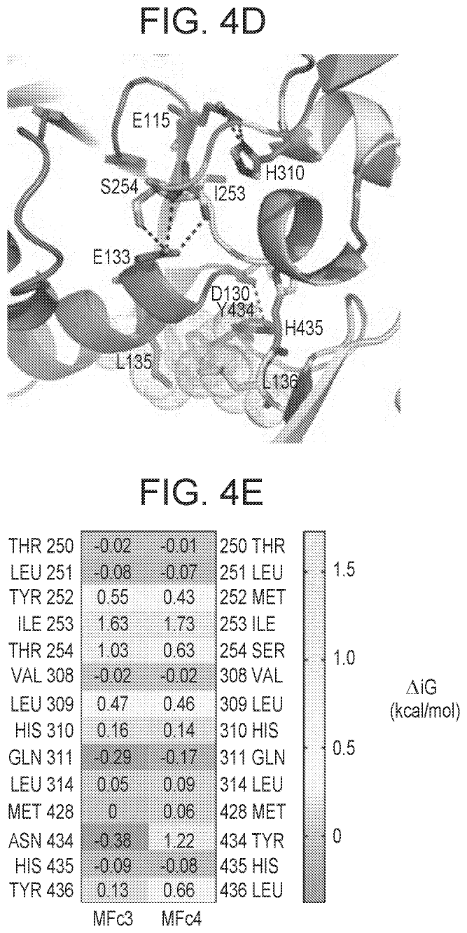

In keeping with our goal of building a set of monomeric Fc variants for modulating FcRn-mediated circulation half-life, we turned our focus to functional and structural characterization of the FcRn interaction with our monomeric Fc molecules. Using recombinant FcRn binding analysis, we found that MFc3 differed significantly from MFc1, showing an equilibrium dissociation constant (K_{D}) of approximately 300 nM (Table A). This finding suggested that the S354E substitution did not alter the interaction with FcRn. To confirm this mode of binding, the MFc3/FcRn complex was prepared at low pH and was subsequently purified and crystallized, and diffraction data were collected to a resolution of 2.6 Å (FIG. 4a). The closest Fc-FcRn complex structure available for comparison was the one solved by our group, which consists of human IgG1 Fc (with the YTE set of mutations) bound to human FcRn in complex with human serum albumin at 3.8 Å (PDB ID: 4N0U).²⁹ Comparison of the two interfaces showed that the mode of interaction remained nearly identical, having minor differences that most likely arose from the difference in resolving the side chains in the electron density maps. This structural and functional invariance of the Fc-FcRn interaction was expected because the S354E mutation is more than 20 Å away from the FcRn interface.

TABLE A
Equilibrium binding of monomeric Fc variants to
human FcRn in a recombinant 1:1 binding format

| Construct | K_{D} (nM) at pH 6 |
|---|---|
| MFc1 (C4) | 370 |
| MFc2 (C4n) | 3,560 |
| MFc3 | 330 |
| MFc4 | 5 |
| IgG1 Fc | 300 |

To evaluate whether the S354E mutation could indeed be used to bring T1 to a monomeric state, we crystallized T1-S354E (MFc4) in complex with FcRn (FIG. 4b). The resolved MFc4/FcRn complex structure demonstrated that MFc4 was indeed monomeric. Despite the fact that the crystallization condition search for the MFc3/FcRn and MFc4/FcRn complexes was done independently, the crystals grew from the same condition and demonstrated nearly identical cell parameters and space group. As expected from previous work, the sequence changes from MFc3 to MFc4 resulted in a significant increase in recombinant FcRn binding affinity, from 300 nM to 5 nM (Table A).²⁵ pH-dependent FcRn binding was observed in these molecules, showing ˜50-fold decrease from pH 6 binding to neutral pH binding (data not shown). The overall structures of the MFc4/FcRn and MFc3/FcRn complexes superimpose with an RMSD of 0.35 Å over 3,700 non-hydrogen atoms, suggesting a high degree of similarity. A comparative view of the MFc3/FcRn and MFc4/FcRn complex structures revealed distinct details of the Fc-FcRn interaction and provided structural explanations for the differences we observed in FcRn binding. The newly introduced Tyr434 and Leu436 in MFc4 increased the interface area from 500 Å² to nearly 600 Å², while adding some hydrophobicity to the interaction (FIG. 4c, 4d). Residue Leu135 in FcRn, which had been known to be a contributor of hydrophobicity at the Fc/FcRn interface,²⁹ was now more involved in the presence of the Tyr434 and Leu436 in MFc4.

Using PDBePISA analysis, we captured the energy contributions from individual residues in the binding pockets of MFc3/FcRn versus MFc4/FcRn with a differential solvation energy heat map (FIG. 4e). As a measurement of the extent of the binding interface, the differential solvation energy calculations reflect the part of the structure's surface that becomes inaccessible to solvent.^{30,31} The strongest contributors to the binding interface from MFc4 were found to be Ile253, Tyr434, and Leu436, in contrast to residues Ile253 and Thr254 in MFc3. The total solvation energy change was 5.09 kcal/mol for MFc4, a notably stronger hydrophobic interaction than MFc3, which had a total AiG of 3.14 kcal/mol (FIG. 4e). As a comparison, we ran the same calculation on the only other available human YTE IgG1 Fc-FcRn complex and found a similar AiG of 2.67 kcal/mol, which was consistent with the binding affinity measurements.

The MFc4/FcRn complex structure also provided us with structural insight into the wild-type human Fc/FcRn interaction, which had never been available before for two reasons. First, wild-type human Fc/FcRn interaction is relatively weak, making complex purification difficult, if not impossible. Second, the propensity of dimeric Fc for crystallization (hence the name “fragment crystallizable”) is so high that most attempts yield crystals containing just Fc (unpublished data). Prior to the availability of our stable monomeric Fc variants, we had to rely on a rat Fc or FcYTE for improved affinity between Fc and FcRn, along with albumin to disrupt the Fc crystal lattice formation, to increase the residence time of the bound state for crystal formation.²⁹ Now, with the help of MFc3 and MFc4 structural complexes with FcRn, with their two distal sets of mutations, we were able to gain a better understanding between wild-type binding interfaces and their mutation sets. For example, at the wild-type interface around residues 252, 254, and 256, where the YTE (M252Y/S254T/T256E) mutations reside, the interface solvation energy map showed that Met252 had only moderate contribution to the FcRn interface, whereas Thr256 was not actually involved (FIG. 4e). The most significant FcRn interaction from the 252-254-256 loop was provided by Ser254. This analysis also explains why the YTE set of mutations could improve affinity between Fc and FcRn and why the strongest contribution came from its S254T substitution.

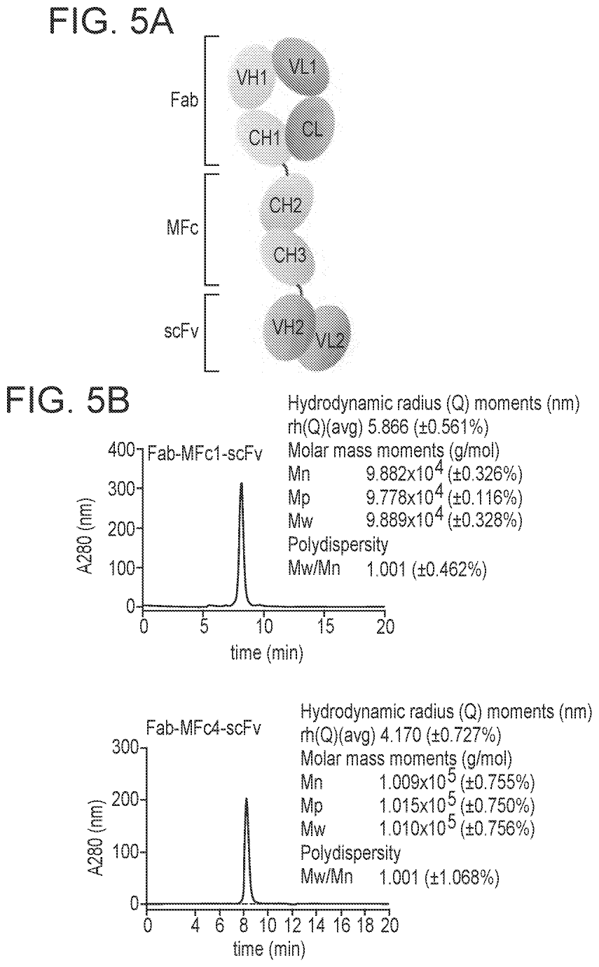

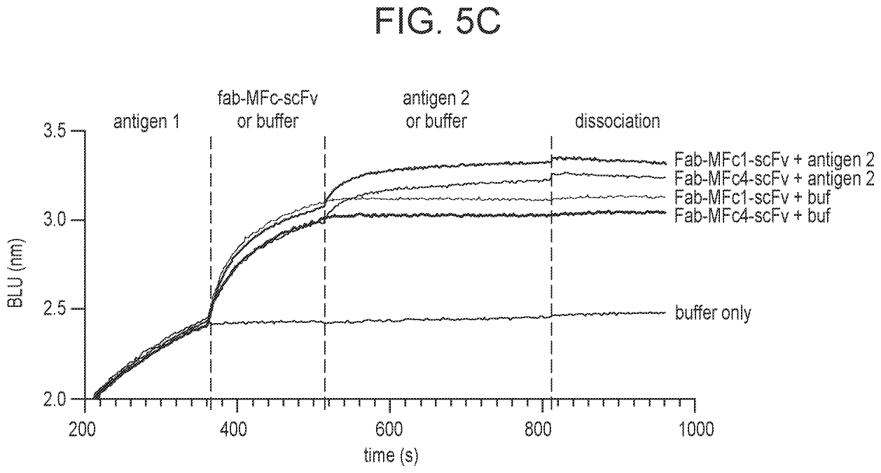

Construction of a monomeric bispecific molecule. These monomeric Fc molecules can be easily used as building blocks for designing monovalent, dual-targeting Fc fusion proteins. Previously we used the MFc1 variant to generate an onartuzumab Fab-MFc1 fusion protein.^{8,13} In the present study, we designed the first example of monovalent bispecific targeting molecules with the monomeric Fc constructs. With a single-plasmid construction, we attached the same Fab domain from onartuzumab to the N-terminus of MFc1 or MFc4, along with a C-terminal single-chain variable fragment (scFv) of an antibody targeting programmed cell death ligand 1 (PD-L1) (FIG. 5a). The constructs were transfected for transient expression in HEK293 suspension cultures (expression titers around 90 mg/L), and the protein was subsequently purified in a single-step protein A purification. SEC-MALS analysis suggested that the protein was monodisperse and had the expected molecular weight of 100 kDa (FIG. 5b). The dual-targeting activity of the monomeric bispecific molecules was confirmed on an Octet platform in a sandwich format (FIG. 5c). The bispecific molecules also maintained their corresponding FcRn binding at pH 6 within 2- to 3-fold of that of the Fc domain binding alone.

Improved in vivo half-life with next-generation monomeric Fc. We achieved significant improvement of in vitro FcRn binding in the MFc4 variant as compared with MFc1. We also wanted to evaluate whether this improvement would translate into enhancement of in vivo serum half-life. The newly generated Fab-MFc4-scFv and Fab-MFc1-scFv, with molecular mass (100 kDa) well above the typical renal filtration clearance size (˜60 kDa),³⁴ are ideal molecules to evaluate the implications of improving the in vivo half-life of FcRn binding. We carried out in vivo pharmacokinetic (PK) studies in hemizygous human FcRn (TG276) transgenic mice. This mouse model is a well-studied model that reflects demonstrable PK impact on human FcRn binding from Fc mutations and a standard IgG1 with a serum half-life of approximately 18 hours.^{13,25,35} Mice were dosed with fusion proteins at 2.5 mg/kg, and serum protein concentrations were determined by enzyme-linked immunosorbent assay (ELISA).

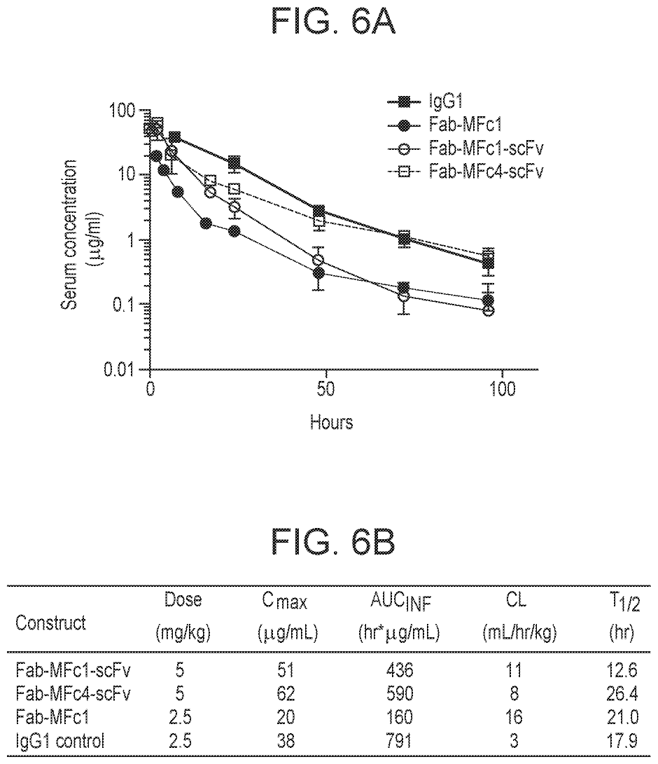

The Fab-MFc4-scFv bispecific protein had higher serum levels than Fab-MFc1-scFv (FIG. 6a). PK parameters were analyzed and determined, showing that the clearance rate and terminal half-life for Fab-MFc4-scFv were significantly greater than those of Fab-MFc1-scFv, by nearly twofold. This indicates that the stronger MFc4-mediated human FcRn binding contributes to enhanced serum protein recycling compared with MFc1. As expected, the increased molecular size contributed to reduced clearance from Fab-MFc1 to the bispecific molecule Fab-MFc1-scFv.

### Discussion

Monovalent antibodies or fusion proteins based on monomeric Fc have the potential to confer IgG-like serum properties to an expanded class of protein therapeutics. Following the successful engineering of a stable monomeric Fc, we took on the challenge to build and expand the utility of the MFc platform to achieve several key properties.

First, we wanted to establish a more universal monomeric Fc molecule that would accommodate alternative Fc mutations for half-life tuning, including the potential for further half-life extension.

Second, we sought to demonstrate that the MFc platform could indeed sustain the monomeric state and be stable for bispecific molecular targeting, a desired targeting strategy to enable novel therapeutic applications including immuno-oncology and receptor-mediated transcytosis.^{4-7,9} A monovalent bispecific drug format built around a monomeric Fc could offer the advantages of a complete ablation of effector function and reduction of toxicity and off-target sink, a design feature that is optimal for T-cell and other immune-cell engagers. Finally, we aimed to validate a computational approach to enable the development of future monomeric Fc designs. To accomplish these goals, we attempted harnessing the power of structure-guided molecular design.

We had previously shown that the YTE mutation in MFc1 was able to improve FcRn binding affinity to offset the reduction in binding avidity.¹³ Based on output from a prior Fc phage library and engineering work, we identified a mutation set, T1 (FIG. 1), with promising enhancement in FcRn binding at pH 6.0. This offered us an ideal test case to evaluate the adaptability of the monomer-stabilizing mutations to half-life mutations in the CH3 domain, as opposed to the YTE mutations from the CH2 domain.

The crystal structure of the T1 protein suggested that the engineered Fc could be engaged in a newly packed dimer formation at high protein concentrations. Although we did not find any direct contribution from the new set of half-life extension mutations on this new form of Fc dimerization, we believe that those mutations play a role in allowing for an induced dimerization under tight packing conditions. A close examination of this dimer interface guided our rational design to enlarge the side chain of residue at position 354. From the set of mutations to replace the serine residue, we chose glutamic acid based on its superior thermal stability profile (FIG. 2). Using solution analytical methods and crystallography, we demonstrated that S354E was able to sustain a monomeric Fc structure, and the glutamic acid side chain protruded out as designed (FIG. 3). The confirmation of the adaptability and activity of S354E for monomeric Fc structures was observed in co-crystal structures when MFc3 and MFc4 maintained the monomeric state and retained extensive engagement with FcRn (FIG. 4a, 4c). This is the first time it has been demonstrated that co-crystal structures can be easily generated to interrogate the Fc-FcRn interface and that quantifiable differences exist in those binding interactions. Using a differential solvation energy calculation for the binding interface, we were able to quantify the enhanced FcRn binding due to extended hydrophobic interaction in MFc4 (FIG. 4b-e).

The availability of monomeric Fc molecules with variable FcRn binding capabilities allowed us to validate the use of the MFc platform for building monovalent bispecific molecular targeting. We generated Fab-MFc-scFv molecules with both MFc1 and MFc4 that had demonstrable monomer conformational purity and dual-targeting activity. With a molecular size well above the renal filtration cutoff, these molecules are well-suited for evaluating the in vivo pharmacokinetic consequences from improving FcRn binding. We found that the Fab-MFc4-scFv bispecific molecule sustained a higher serum level than Fab-MFc1-scFv, nearing that of a standard IgG1 antibody. These results also indicate that these MFc constructs can contribute to tunable serum protein recycling and can provide a versatile bispecific platform for the development of next-wave technology to support therapeutic advances.

The exposure of the CH3 domain due to the disruption of the Fc dimer is not a new phenomenon. IgG4 backbone had been chosen for our MFc platform due to the ready engagement of Fab-arm exchange of the IgG4 molecules which alternates the Fc between a dimer-monomer equilibrium.³⁶ In addition, to launch a strong and specific immune response, T-cell epitopes need to be processed and presented by antigen presenting cells.³⁷ To mitigate the concern that the newly introduced mutations may form a novel T cell epitope, an in silico T cell epitope prediction around the monomer-forming mutations in MFc1 and MFc4 was performed. We observed an overall low predicted binding rate for these mutations, indicating a lower immunogenicity risk.³⁸

The ultimate success of curative therapeutics will rely on many collaborative efforts to address the balances of potency versus toxicity and serum half-life versus tissue penetration, in conjunction with a deepened understanding of immunity and translational sciences. With the ability and flexibility to present monovalent bispecific targeting motifs to sidestep any undesired Fc receptor-mediated cytotoxicity and off-target sinks while mimicking IgG-like serum properties, the MFc platform presents a timely possibility to further expand the exploration of immune-cell engagers.

### Methods

Ethics statement. The protocol (MI-13-0012) requiring the use of animals in these studies was reviewed and approved by AstraZeneca's Institutional Animal Care and Use Committee and complies with the animal welfare standards of the U.S. Department of Agriculture, the Guide for the Care and Use of Laboratory Animals, and the Association for Assessment and Accreditation of Laboratory Animal Care.

Antibody cloning, expression, and purification. All antibody positions are listed according to the Kabat numbering convention for the variable domains and EU numbering convention for the CH2-CH3 domain.^{41,42} All chemicals were of analytical grade. Oligonucleotides were purchased from Eurofins MWG Operon (Louisville, KY). A plasmid encoding mAb-J was generated with the In-Fusion HD cloning kit from Takara Bio (Mountain View, CA), encoding variable heavy chain and variable light chain sequences into an in-house IgG1 mammalian expression vector. Point mutations were introduced by site-directed mutagenesis, using the QuikChange Multi Lightning mutagenesis kit (Agilent Technologies, Santa Clara, CA).

The variants were transiently transfected into the human embryonic kidney cell line HEK293FT, using 293Fectin transfection reagent (Life Technologies, Carlsbad, CA). Cells were grown in FreeStyle 293-F Expression Medium (Life Technologies). The expressed antibodies were purified from cell supernatant by affinity chromatography, with a HiTrap Protein A column (GE Healthcare Life Sciences, Marlborough, MA). Antibody was eluted with Pierce IgG Elution Buffer (Thermo Fisher Scientific, Waltham, MA) and neutralized with 1 M Tris, pH 8.0. Antibodies were dialyzed into phosphate buffered saline (PBS), pH 7.2. Monomer content for all the antibodies was determined by analytical SEC to be greater than 95%.

SEC-MALS and analytical ultracentrifugation. Purified Fc clones and fusion proteins at concentrations of 1 mg/mL or greater were analyzed by SEC, using a TSK-GEL G2000SWXL column with a 14-mL bed volume (Tosoh Biosciences, Tokyo, Japan) on an 1100 HPLC instrument (Agilent, Santa Clara, CA) at room temperature. The samples were eluted isocratically in PBS at a flow rate of 1 mL/min for 20 min. Eluted proteins were detected with ultraviolet absorbance at a wavelength of 280 nm. Data analysis was performed with ChemStation software (version A.02.10). Column calibration was performed with a set of molecular-weight standards ranging from 10 to 500 kDa (Bio-Rad, Hercules, CA). In-line SEC-MALS was performed. Sample measurements were performed on a Dawn Heleos II MALS with an Optilab Rex refractometer (Wyatt Technologies, Santa Barbara, CA). The molecular mass of each protein within a defined chromatographic peak was calculated by using Astra, version 6.1 (Wyatt Technologies).

For analytical ultracentrifugation analysis, samples and reference buffer were loaded into 12-mm double-sector cells with Epon centerpieces, then placed in an An-50 Ti rotor for ultracentrifugation at 50,000 rpm with an Optima XL-I centrifuge set to 20° C. (Beckman-Coulter, Indianapolis, IN). The sedimentation data collected at 280 nm for scans 2 to 160 were analyzed with Sedfit software (version 16.1c) to generate c(s) distributions.^{43,44} The partial specific volume was set to 0.73 mL/g. Solution density and viscosity values for PBS were set to 1.00523 g/mL and 1.019 mPa s, respectively, using the calculated value from the Sednterp program (version 20130813).⁴⁵ Based on the Svedberg equation, a monomeric Fc with a molecular mass of 27 kDa is expected to have a sedimentation coefficient of 1.7-2.4 S (Svedberg units), assuming a frictional ratio of 1.3-1.8 (globular to extended shape).

Crystallization, data collection, and structure determination. Prior to crystallization, protein A purified T1, MFc3, and MFc4 were further purified by ion exchange chromatography on a Q HP 5 mL prepacked column (GE Healthcare Life Sciences) equilibrated with 25 mM Tris-HCl buffer at pH 8 further purified by SEC, using a Superdex 200 Increase 10/300 GL column (GE Healthcare Life Sciences) pre-equilibrated with 25 mM Tris-HCl, pH 8, and 100 mM NaCl. The cultured media of recombinant heterodimeric FcRn after harvest was pH adjusted for affinity purification on an IgG Sepharose column (GE Healthcare Life Sciences). After FcRn was purified on a Q HP column (GE Healthcare Life Sciences), it was dialyzed into 30 mM sodium acetate buffer at pH 5.2 and complexed with MFc3 and MFc4 at a 1% molar deficit of FcRn, and the complex was purified by SEC using the same Superdex 200 column equilibrated with 30 mM sodium acetate, pH 5.2, and 100 mM NaCl. Complex composition was confirmed by SDS PAGE.

Initial crystallization trials for all proteins and protein complexes were carried out by the sitting-drop vapor-diffusion method at 20° C. The crystallization drops were dispensed in 96-well crystallization plates (Intelli-plate 102-0001-20; Art Robbins Instruments, Sunnyvale, CA) by a Phoenix robot (Art Robbins Instruments) and were composed of equal volumes of protein and reservoir buffer. For crystallization of T1 and MFc3 by themselves, we used commercially available screens (Hampton Research, Aliso Viejo, CA; Molecular Dimensions, Suffolk, UK). For the crystallization of the FcRn-complexed proteins, we generated a new screen consisting of a combination of the low-pH conditions contained in commercially available screens. Diffraction quality crystals were grown in the crystallization optimization step in hanging drop format from the following crystallization solutions: T1: 0.01 M zinc sulfate heptahydrate; 0.1 M morpholineethanesulfonic acid (MES) monohydrate, pH 6.5, and 25% (w/v) PEG 550 MME at a protein concentration of 5.5 mg/mL. MFc4/FcRn complex: 0.2 M magnesium chloride hexahydrate, 1 M sodium iodide, 0.1 M MES, pH 6 and 20% PEG 6000 at a protein concentration of 6.35 mg/mil MFc3/FcRn complex: 0.2 M magnesium chloride hexahydrate, 30% 1,5-diaminopentane dihydrochloride, 0.1 M MES, pH 6 and 20% PEG 6000 at a protein concentration of 6 mg/mL. The crystals for MFc3 were harvested directly from the original sitting drop plates from a condition consisting of 0.8% anesthetic alkaloids (2% w/v lidocaine hydrochloride monohydrate, 2% w/v procaine hydrochloride, 2% w/v proparacaine hydrochloride, 2% w/v tetracaine hydrochloride), 0.1 M MOPS (acid) and sodium HEPES pH 7.5, and a 50% v/v mix of precipitants (40% v/v ethylene glycol, 20% w/v PEG 8000) at a protein concentration of 7 mg/mL. All crystals harvested for X-ray analysis were flash-cooled by dipping in liquid nitrogen Diffraction data were collected from single crystals on beamline BL9-2 of a Stanford Synchrotron Radiation Lightsource equipped with a Pilatus 6M PAD detector (Paul Scherer Institute, Villigen, Switzerland) over an oscillation range of 180°, an increment of 0.5°, and a 0.8-s exposure per image. Diffraction data were processed with the XDS program.⁴⁶ All crystallographic calculations were carried out with the CCP4 software suite (version 7.0).⁴⁷ The molecular replacement procedure was performed by using the Molrep program.⁴⁸ Structure refinement was performed with Refmac5, and model adjustments were carried out with the “O” program.^{49,50} Figures with structures were generated with PyMOL (Schrödinger, New York, NY).

Octet binding analysis. Binding measurements of the monomeric Fc and its fusion proteins to in-house purified recombinant human FcRn were carried out by biolayer interferometry on an Octet384 instrument (ForteBio, Menlo Park, CA). Biotinylated FcRn at 1 μg/mL in PBS buffer (pH 7.4) or 100 mM MES buffer (pH 6.0), with 3 mg/mL bovine serum albumin, 0.05% (vol/vol) and Tween 20 (1× Kinetics Buffer; ForteBio), were captured on streptavidin biosensors (ForteBio). The loaded biosensors were washed with assay buffer to remove any unbound protein, followed by association and dissociation measurements with serial dilutions of the different Fc variants or Fc fusion constructs. Octet software (version 7.2) was used to calculate kinetic parameters (kₒₙ and k_{off}) and apparent affinities (K_{D}) from a nonlinear fit based on the 1:1 binding kinetic model of the data, with the equation

$$( A + B {↔}_{{k}_{off}}^{{k}_{on}} AB ) .$$

Concurrent binding measurements of Fab-MFc-scFv molecules to recombinant antigen proteins were also performed. Biotinylated cMet protein was captured at 5 μg/mL on streptavidin biosensors (ForteBio) in PBS buffer, pH 7.2, with 1× kinetics buffer. The binding steps included 300 nM Fab-MFc-scFv with buffer control, followed by binding to antigen 2 with buffer control.

In vivo PK in hFcRn transgenic mice. Human FcRn transgenic mice used in this study are the F1 cross of murine FcRn-deficient B6.129X1-FcgrttmlDcr/DcrJ and human FcRn cDNA transgenic line B6.Cg-FcgrttmlDcr Tg (CAG-FCGRT) 276 Dcr/DcrJ. Sex-matched (6-16-week-old) mice were given a bolus intravenous dose of 2.5 mg/kg monomeric Fc fusion proteins on day 0. Eight mice were used per protein, and two groups of mice (groups A and B) were bled at alternate time points. Blood samples were obtained from the retroorbital plexus with capillary pipettes at different time points throughout the 2-3-week-long study. All animals remained healthy throughout the study. A quantitative ELISA was used to monitor the serum concentrations of the tested antibodies. Briefly, 96-well plates were coated with 2 μg/mL cMet extracellular domain. The plates coated with 5 μg/mL cMet were incubated overnight at 4° C., blocked with 3% bovine serum albumin in PBS-Tween, and then incubated with the diluted serum samples at different time points. Goat antihuman Fc-specific horseradish peroxidase-conjugated antibody at 1:10⁴ dilution (Jackson ImmunoResearch Laboratories, West Grove, PA) was used for detection. Absorbance at 450 nm was measured after development with 3,3′,5,5′-tetramethylbenzidine substrate (KPL, Gaithersburg, MD) according to the manufacturer's directions. Standard curves were generated for each antibody variant. The linear portions of standard curves were generated in Prism (version 6; GraphPad Software, La Jolla, CA) and then used to quantify human anti-cMet fusion proteins in the serum samples. Non-compartmental PK data analysis was performed with Phoenix 64 WinNonlin 6.3 (Pharsight, Mountain View, CA). The maximum observed peak plasma concentration was determined by inspection of the observed data using WinNonlin. The terminal elimination half-life was determined with the equation ln(2)/λz, where λz is the slope of the terminal portion of the natural-log concentration-time curve, determined by linear regression of at least the last three time points. The systemic exposure was determined by calculating the area under the curve (AUC) for the plasma concentration versus time graph (AUCₗₐₛₜ) from the start of dosing to the time of last measurable concentration, using the linear/log trapezoidal rule. AUC for the plasma-concentration versus time graph from time 0 to infinity (AUC_{∞}) was calculated as: AUCₗₐₛₜ+C_{last/λz}, where Cₗₐₛₜ is the last quantifiable concentration. Clearance (CL) was calculated by dose/AUC_{∞}, and steady-state volume of distribution was calculated as: (AUMC_{∞}×CL)/AUC_{∞}, where AUMC_{∞} is the AUC from the first moment extrapolated to infinity. PK parameters were summarized statistically and presented as mean.

### REFERENCES

1. Kaplon, H. & Reichert, J. M. Antibodies to watch in 2019. MAbs 11, 219-238 (2019).
2. Hay, M., Thomas, D. W., Craighead, J. L., Economides, C. & Rosenthal, J. Clinical development success rates for investigational drugs. Nat Biotechnol 32, 40-51 (2014).
3. Wolf, E., Hofmeister, R., Kufer, P., Schlereth, B. & Baeuerle, P. A. BiTEs: bispecific antibody constructs with unique anti-tumor activity. Drug Discov Today 10, 1237-44 (2005).
4. Felices, M., Lenvik, T. R., Davis, Z. B., Miller, J. S. & Vallera, D. A. Generation of BiKEs and TriKEs to improve NK cell-mediated targeting of tumor cells. Methods Mol Biol 1441, 333-46 (2016).
5. Vallera, D. A. et al. IL15 trispecific killer engagers (TriKE) make natural killer cells specific to CD33+ targets while also inducing persistence, in vivo expansion, and enhanced function. Clin Cancer Res 22, 3440-50 (2016).
6. Suurs, F. V., Lub-de Hooge, M. N., de Vries, E. G. E. & de Groot, D. J. A. A review of bispecific antibodies and antibody constructs in oncology and clinical challenges. Pharmacol Ther 201, 103-119 (2019).
7. Labrijn, A. F., Janmaat, M. L., Reichert, J. M. & Parren, P. Bispecific antibodies: a mechanistic review of the pipeline. Nat Rev Drug Discov 18, 585-608 (2019).
8. Merchant, M. et al. Monovalent antibody design and mechanism of action of onartuzumab, a MET antagonist with anti-tumor activity as a therapeutic agent. Proc Natl Acad Sci USA 110, E2987-96 (2013).
9. Niewoehner, J. et al. Increased brain penetration and potency of a therapeutic antibody using a monovalent molecular shuttle. Neuron 81, 49-60 (2014).
10. Harwood, S. L. et al. ATTACK, a novel bispecific T cell-recruiting antibody with trivalent EGFR binding and monovalent CD3 binding for cancer immunotherapy. Oncoimmunology 7, e1377874 (2017).
11. Mazor, Y. et al. Improving target cell specificity using a novel monovalent bispecific IgG design. mAbs 7, 377-89 (2015).
12. Klein, C., Schaefer, W. & Regula, J. T. The use of CrossMAb technology for the generation of bi- and multispecific antibodies. mAbs 8, 1010-20 (2016).
13. Shan, L. et al. Generation and Characterization of an IgG4 Monomeric Fc Platform. PLoS One 11, e0160345 (2016).
14. Zhou, L. et al. Single chain Fc-dimer-human growth hormone fusion protein for improved drug delivery. Biomaterials 117, 24-31 (2017).
15. Strohl, W. R. Fusion Proteins for Half-Life Extension of Biologics as a Strategy to Make Biobetters. BioDrugs 29, 215-39 (2015).
16. Strohl, W. R., Strohl, L. M. Therapeutic antibody engineering: current and future advances driving the strongest growth area in the pharma industry, (Camdrige, 2012).
17. Rath, T. et al. Fc-fusion proteins and FcRn: structural insights for longer-lasting and more effective therapeutics. Crit Rev Biotechnol 35, 235-54 (2015).
18. Liu, L. Pharmacokinetics of monoclonal antibodies and Fc-fusion proteins. Protein Cell 9, 15-32 (2018).
19. Ying, T. et al. Engineered antibody domains with significantly increased transcytosis and half-life in macaques mediated by FcRn. mAbs 7, 922-30 (2015).
20. Wilkinson, I. C. et al. Monovalent IgG4 molecules: immunoglobulin Fc mutations that result in a monomeric structure. MAbs 5, 406-17 (2013).
21. Ishino, T. et al. Engineering a monomeric Fc domain modality by N-glycosylation for the half-life extension of biotherapeutics. J Biol Chem 288, 16529-37 (2013).
22. Ghetie, V. et al. Increasing the serum persistence of an IgG fragment by random mutagenesis. Nat Biotechnol 15, 637-40 (1997).
23. Roopenian, D. C. & Akilesh, S. FcRn: the neonatal Fc receptor comes of age. Nat Rev Immunol 7, 715-25 (2007).
24. Dall'Acqua, W. F. et al. Increasing the affinity of a human IgG1 for the neonatal Fc receptor: biological consequences. J Immunol 169, 5171-80 (2002).
25. Borrok, M. J. et al. pH-dependent binding engineering reveals an FcRn affinity threshold that governs IgG recycling. J Biol Chem 290, 4282-90 (2015).
26. Mackness, B. C. et al. Antibody Fc engineering for enhanced neonatal Fc receptor binding and prolonged circulation half-life. mAbs 11, 1276-1288 (2019).
27. Martin, W. L., West, A. P., Jr., Gan, L. & Bjorkman, P. J. Crystal structure at 2.8 Å of an FcRn/heterodimeric Fc complex: mechanism of pH-dependent binding. Mol Cell 7, 867-77 (2001).
28. Martin, W. L. & Bjorkman, P. J. Characterization of the 2:1 complex between the class I MHC-related Fc receptor and its Fc ligand in solution. Biochemistry 38, 12639-47 (1999).
29. Oganesyan, V. et al. Structural insights into neonatal Fc receptor-based recycling mechanisms. J Biol Chem 289, 7812-24 (2014).
30. Viswanath, S., Ravikant, D. V. & Elber, R. Improving ranking of models for protein complexes with side chain modeling and atomic potentials. Proteins 81, 592-606 (2013).
31. Krissinel, E. & Henrick, K. Inference of macromolecular assemblies from crystalline state. J Mol Biol 372, 774-97 (2007).
32. Iacob, R. E. et al. Investigating monoclonal antibody aggregation using a combination of H/DX-MS and other biophysical measurements. J Pharm Sci 102, 4315-29 (2013).
33. Ionescu, R. M., Vlasak, J., Price, C. & Kirchmeier, M. Contribution of variable domains to the stability of humanized IgG1 monoclonal antibodies. J Pharm Sci 97, 1414-26 (2008).
34. Tang, L., Persky, A. M., Hochhaus, G. & Meibohm, B. Pharmacokinetic aspects of biotechnology products. J Pharm Sci 93, 2184-204 (2004).
35. Haraya, K., Tachibana, T., Nanami, M. & Ishigai, M. Application of human FcRn transgenic mice as a pharmacokinetic screening tool of monoclonal antibody. Xenobiotica 44, 1127-34 (2014).
36. Rispens, T., Ooijevaar-de Heer, P., Bende, O. & Aalberse, R. C. Mechanism of immunoglobulin G4 Fab-arm exchange. J Am Chem Soc 133, 10302-11 (2011).
37. Parker, D. C. T cell-dependent B cell activation. Annu Rev Immunol 11, 331-60 (1993).
38. Jawa, V. et al. T-Cell Dependent Immunogenicity of Protein Therapeutics Pre-clinical Assessment and Mitigation-Updated Consensus and Review 2020. Front Immunol 11, 1301 (2020).
39. Hegde, P. S. & Chen, D. S. Top 10 Challenges in Cancer Immunotherapy. Immunity 52, 17-35 (2020).
40. Oganesyan, V. et al. Structural insights into the interaction of human IgG1 with FcgammaRI: no direct role of glycans in binding. Acta Crystallogr D Biol Crystallogr 71, 2354-61 (2015).
41. Edelman, G. M. et al. The covalent structure of an entire yG immunoglobulin molecule. Proc Natl Acad Sci USA 63, 78-85 (1969).
42. Kabat, E. A., Te Wu, T., Foeller, C., Perry, H. M. & Gottesman, K. S. Sequences of Proteins of Immunological Interest, (National Institutes of Health, U. S. Department of Health and Human Services, Bethesda, MD, 1991).
43. Brown, P. H., Balbo, A. & Schuck, P. Characterizing protein-protein interactions by sedimentation velocity analytical ultracentrifugation. Curr Protoc Immunol Chapter 18, Unit 18.15 (2008).
44. Schuck, P. Size-distribution analysis of macromolecules by sedimentation velocity ultracentrifugation and lamm equation modeling. Biophys J 78, 1606-19 (2000).
45. Lane, T. M., Shah, B. D., Ridgeway, T. M. & Pelletier, S. L. Computer-aided interpretation of analytical sedimentation data for proteins. in Analytical Ultracentrifugation in Biochemistry and Polymer Science (eds. Harding, S. E., Rowe, A. J. & Horton, J. C.) 90-125 (The Royal Society of Chemistry, Cambridge, 1992).
46. Kabsch, W. Xds. Acta Crystallogr D Biol Crystallogr 66, 125-32 (2010).
47. Winn, M. D. et al. Overview of the CCP4 suite and current developments. Acta Crystallogr D Biol Crystallogr 67, 235-42 (2011).
48. Vagin, A. & Teplyakov, A. Molecular replacement with MOLREP. Acta Crystallogr D Biol Crystallogr 66, 22-5 (2010).
49. Jones, T. A., Zou, J. Y., Cowan, S. W. & Kjeldgaard, M. Improved methods for building protein models in electron density maps and the location of errors in these models. Acta Crystallogr A 47 (Pt 2), 110-9 (1991).
50. Murshudov, G. N. et al. REFMAC5 for the refinement of macromolecular crystal structures. Acta Crystallogr D Biol Crystallogr 67, 355-67 (2011).

### Sequences

mFc4
(SEQ ID NO: 1)
GPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEK
TISKAKGQPREPQVYTFPPEQEEMTKNQVSLRCLVKGFYPSDIAVEWES
NGQPENNYKTTKPVLDSDGSFRLESRLTVDKSRWQEGNVFSCSVMHEAC
SYHLCKSLSLSL

1. A fusion protein comprising a FcRn binding fragment of an Fc region of an IgG molecule, wherein the FcRn binding fragment comprises:
a. F at position 351
b. R, K, D, E, F, Y, P, G, L or M at position 354
c. R at position 366
d. K at position 395
e. R at position 405; and
f. E at position 407
wherein the amino acid numbering is according to the EU index.

2. A polypeptide comprising a FcRn binding fragment of an Fc region of an IgG molecule, wherein the FcRn binding fragment comprises:
a. F at position 351
b. R, K, D, E, F, Y, P, G, L or M at position 354
c. R at position 366
d. K at position 395
e. R at position 405; and
f. E at position 407
wherein the amino acid numbering is according to the EU index.

3. A molecule comprising a non-protein agent conjugated to a FcRn binding fragment of an Fc region of an IgG molecule, wherein the FcRn binding fragment comprises:
a. F at position 351
b. R, K, D, E, F, Y, P, G, L or M at position 354
c. R at position 366
d. K at position 395
e. R at position 405; and
f. E at position 407
wherein the amino acid numbering is according to the EU index.

4. The fusion protein of claim 1, polypeptide of claim 2 or molecule of claim 3, wherein the FcRn binding fragment comprises R, K, D or E at position 354, optionally D or E, wherein the amino acid numbering is according to the EU index.

5. The fusion protein, polypeptide or molecule of any preceding claim, wherein the FcRn binding fragment comprises E at position 354, wherein the numbering is according to the EU index.

6. The fusion protein, polypeptide or molecule of any preceding claim, wherein the FcRn binding fragment comprises from about amino acid residue 216 to about amino acid residue 446 of an IgG molecule, wherein the numbering is according to the EU index.

7. The fusion protein, polypeptide or molecule of any preceding claim, wherein the FcRn binding fragment comprises from about amino acid residue 236 to about amino acid residue 446 of an IgG molecule, wherein the numbering is according to the EU index.

8. The fusion protein, polypeptide or molecule of any preceding claim, wherein the IgG molecule is an IgG4.

9. The fusion protein, polypeptide or molecule of any preceding claim, wherein the FcRn binding fragment further comprises half-life extension mutations.

10. The fusion protein, polypeptide or molecule of claim 9, wherein the FcRn binding fragment comprises a Y at position 252, a T at position 254 and a E at position 256, wherein numbering is according to the EU index.

11. The fusion protein, polypeptide or molecule of claim 9, wherein the Fc region comprises the following amino acids:
a. C at positions 432 and 437;
b. S, H, R, P, T, K, A, M or N at position 433;
c. Y, N, R, W, H, F, S, M or T at position 434;
d. H at position 435; and
e. L, Y, F, R, I, K, M, V, H, S or T at position 436
wherein numbering is according to the EU index.

12. The fusion protein, polypeptide or molecule of claim 11, wherein the FcRn binding fragment comprises:
a. Y, R, W, H or F at position 434; and
b. L, R, I, K, M, V or H at position 436
wherein numbering is according to the EU index.

13. The fusion protein, polypeptide or molecule of claim 12, wherein the FcRn binding fragment comprises the following amino acids:
a. C at positions 432 and 437;
b. S at position 433;
c. Y at position 434;
d. H at position 435; and
e. L at position 436
wherein numbering is according to the EU index.

14. The fusion protein, polypeptide or molecule of any preceding claim, wherein the FcRn binding fragment comprises deletion of the amino acid at position 438, wherein numbering is according to the EU index.

15. The fusion protein, polypeptide or molecule according to any of claims 1 to 13, wherein Q438 (numbered according to the EU index) of the FcRn binding fragment is deleted.

16. The fusion protein, polypeptide or molecule of any preceding claim, wherein the FcRn binding fragment comprises an E inserted immediately after residue 437, wherein numbering is according to the EU index.

17. The fusion protein or polypeptide of any preceding claim, comprising at least one, such as one or two, antigen binding domains.

18. The fusion protein or polypeptide of any preceding claim, comprising two antigen binding domains.

19. The fusion protein or polypeptide according to any preceding claim, wherein a first antigen binding domain is N-terminal to the FcRn binding fragment.

20. The fusion protein or polypeptide according to any preceding claim, wherein a second antigen binding domain is C-terminal to the FcRn binding fragment.

21. The fusion protein or polypeptide according to any preceding claim, wherein each antigen binding domain is independently selected from a Fv, Fab, Fab′, F(ab′)2, Fab′-SH, diabodies, triabodies, tetrabodies, linear antibodies and scFv.

22. The fusion protein or polypeptide of any preceding claim, comprising a non-IgG protein domain.

23. The fusion protein or polypeptide of claim 22, wherein the non-IgG domain is an immunomodulator, a receptor, a hormone, an enzyme or a drug.

24. The molecule of any of claims 1-16, wherein the non-protein agent is a nucleic acid (e.g., a DNA or an RNA), lipid, glycolipid, polysaccharide, drug, radioisotope, chelated metal, nanoparticle or reporter group, such as fluorescent compounds or compounds which may be detected by NMR or ESR spectroscopy.

25. The molecule of claim 24, wherein the nucleic acid is a DNA, RNA, siRNA, RNAi or microRNA.

26. The molecule of claim 24, wherein the drug is a cytotoxic agent, chemotherapeutic agent, anti-neoplastic agent, anti-angiogenic agent or pro-apoptotic agent.

27. The fusion protein of claim 1, polypeptide of claim 2, or molecule of claim 3, wherein the FcRn binding fragment comprises the amino acid sequence set forth SEQ ID NO: 1.

28. A nucleic acid comprising a nucleotide sequence encoding the fusion protein or polypeptide of any preceding claim.

29. A vector comprising the nucleic acid of claim 28.

30. A host cell comprising the nucleic acid of claim 28 or the vector of claim 29.

31. A nucleic acid comprising a nucleotide sequence encoding the FcRn binding fragment of the molecule of any of claims 1 to 16.

32. A vector comprising the nucleic acid of claim 31.

33. A host cell comprising the nucleic acid of claim 31 or the vector of claim 32.

34. A method of producing the fusion protein or polypeptide of any of claims 1 to 23 or 27, comprising expressing said fusion protein or polypeptide from the host cell of claim 30, and purifying said fusion protein or polypeptide.

35. A pharmaceutical composition comprising the fusion protein, polypeptide or molecule of any of claims 1-27, a pharmaceutically acceptable carrier and a diluent.

36. A pharmaceutical composition according to claim 35, for use in therapy.

37. Use of a pharmaceutical composition according to claim 35, in therapy.

38. A fusion protein, polypeptide or molecule of any of claims 1-27, for use in therapy.

39. Use of a fusion protein, polypeptide or molecule of any of claims 1-27, in therapy.

40. Use of A fusion protein, polypeptide or molecule of any of claims 1-27, in the manufacture of a medicament for the treatment of a disease.

41. A method of treatment comprising administering to a patient in need thereof a therapeutically effective amount of the fusion protein, polypeptide or molecule of any of claims 1-27 or pharmaceutical composition of claim 35.
